(19) United States
(12) Patent Application Publication
(10) Pub. No.: US 2015/0299754 A1
(43) Pub. Date: Oct. 22, 2015

(54) STREPTOMYCES MICROFLAVUS STRAINS AND METHODS OF THEIR USE TO CONTROL PLANT DISEASES AND PESTS

(71) Applicant: Bayer CropScience LP, Research Triangle Park, NC (US)

(72) Inventors: Damian CURTIS, West Sacramento, CA (US); Shaohua GUAN, Pilot Point, TX (US); Magalie GUILHABERT-GOYA, West Sacramento, CA (US); Jonathan S. MARGOLIS, West Sacramento, CA (US); Reed Nathan ROYALTY, West Sacramento, CA (US); Frisby Davis SMITH, West Sacramento, CA (US); Colleen TAYLOR, West Sacramento, CA (US); Hong ZHU, West Sacramento, CA (US)

(21) Appl. No.: 14/688,806

(22) Filed: Apr. 16, 2015

Related U.S. Application Data
(60) Provisional application No. 61/980,526, filed on Apr. 16, 2014.

(51) Int. Cl. C12P 19/40; A01N 43/54

(52) U.S. Cl. CPC C12P 19/40; A01N 43/54

(57) ABSTRACT

The present invention relates to novel strains of Streptomyces microflavus and methods of their use for controlling diseases or pests of a plant. The invention also relates to a fermentation broth obtained by cultivating a gougerotin producing Streptomyces strain, wherein the fermentation broth contains at least about 2 g/L gougerotin. The invention also relates to a method of producing a fermentation broth of a gougerotin producing Streptomyces strain, wherein the fermentation broth contains at least about 2 g/L gougerotin, the method comprising cultivating the Streptomyces strain in a culture medium containing a digestible carbon source and a digestible nitrogen source under aerobic conditions.

### CROSS-REFERENCE TO RELATED APPLICATIONS

The present application claims priority to U.S. Provisional Application No. 61/980,526, filed Apr. 16, 2014, which is hereby incorporated by reference in its entirety.

### FIELD OF INVENTION

The present invention relates to the field of bacterial strains and their ability to control plant diseases and pests.

### BACKGROUND OF INVENTION

Phytophagous mites, especially spider mites, are a major agricultural pest of orchards, greenhouses and many vegetable and fruit crops, including peppers, tomatoes, potatoes, squash, eggplant, cucumber and strawberries. Mites damage leaf and/or fruit surfaces using their sharp mouthparts. Besides direct damage to plant parts (referred to as stippling), mite feeding also causes increased susceptibility to plant diseases.

Mites are acari rather than insects, and few broad spectrum insecticides are also effective against mites. Characteristics of mites and of available miticides pose challenges to mite control. For example, spider mites, one of the most economically important families of mites, generally live on the undersides of leaves of plants, such that they are difficult to treat. Further, mites are known to develop resistance to presently available miticides, many of which have a single mode of action, within two to four years. Few available miticides have activity against mite eggs, making repeat applications necessary. Therefore, there is a need for new miticides having translaminar, ovicidal and strong residual activities in addition to good knockdown activity.

### SUMMARY OF INVENTION

The present invention provides the Streptomyces microflavus strain NRRL B-50550 or a phytophagous-miticidal mutant (strain) derived therefrom. In one embodiment, the phytophagous-miticidal and/or fungicidal mutant strain is Streptomyces microflavus strain M. In another embodiment the phytophagous-miticidal and/or fungicidal mutant strain is Streptomyces microflavus strain NRRL B-50954, Streptomyces microflavus strain NRRL B-50955, Streptomyces microflavus strain NRRL B-50956, Streptomyces microflavus strain NRRL B-50957, or Streptomyces microflavus strain NRRL B-50958. The present invention also provides the Streptomyces puniceus strain A or a phytophagous-miticidal and/or fungicidal mutant (strain) derived therefrom.

The present invention also provides a composition containing Streptomyces microflavus strain NRRL B-50550 or a phytophagous-miticidal and/or fungicidal mutant (strain) derived therefrom. In certain embodiments, the mutant strain is selected from the group consisting of Streptomyces microflavus strain NRRL B-50954, Streptomyces microflavus strain NRRL B-50955, Streptomyces microflavus strain NRRL B-50956, Streptomyces microflavus strain NRRL B-50957, Streptomyces microflavus strain NRRL B-50958, and mutants thereof having all the identifying characteristics of the respective strain. In one aspect, the mutant strain is Streptomyces microflavus strain NRRL B-50958 or a mutant thereof having all the identifying characteristics of the strain.

In one aspect, the composition is a fermentation product of the Streptomyces microflavus strain. The present invention also provides a composition containing Streptomyces puniceus strain A or a phytophagous-miticidal and/or fungicidal mutant (strain) derived therefrom. In one aspect, the composition is a fermentation product of the Streptomyces puniceus strain A or a phytophagous-miticidal and/or fungicidal mutant strain derived therefrom. The present invention also provides a composition containing Streptomyces microflavus strain NRRL B-50954, Streptomyces microflavus strain NRRL B-50955, Streptomyces microflavus strain NRRL B-50956, Streptomyces microflavus strain NRRL B-50957, or Streptomyces microflavus strain NRRL B-50958, or mutants of any one of the aforementioned strains having all the identifying characteristics of the respective strain. In one aspect, the composition is a fermentation product of the Streptomyces microflavus strain NRRL B-50954, Streptomyces microflavus strain NRRL B-50955, Streptomyces microflavus strain NRRL B-50956, Streptomyces microflavus strain NRRL B-50957, or Streptomyces microflavus strain NRRL B-50958.

The present invention also provides a fermentation product obtained by cultivating a gougerotin producing Streptomyces strain, wherein the fermentation product contains at least about 2 g/L gougerotin.

In certain aspects, the method of producing a fermentation broth of a gougerotin-producing Streptomyces strain, wherein the fermentation broth contains at least about 2 g/L gougerotin, comprises: a) screening a collection of Streptomyces strains to identify at least one gougerotin-producing Streptomyces strain; b) generating a plurality of mutant strains from the at least one gougerotin-producing Streptomyces strain; c) screening the plurality of mutant strains to identify at least one mutant strain that produces a fermentation broth containing at least about 2 g/L gougerotin; and d) cultivating the at least one mutant strain in a culture medium containing a digestible carbon source and a digestible nitrogen source under aerobic conditions.

In other aspects, the method of producing a fermentation broth of a gougerotin producing Streptomyces strain, wherein the fermentation broth contains at least about 2 g/L gougerotin, comprises: a) generating a plurality of mutant strains of Streptomyces microflavus NRRL B-50550 and/or Streptomyces microflavus strain No. 091013-02; b) screening the plurality of mutant strains to identify at least one mutant strain that produces a fermentation broth containing at least about 2 g/L gougerotin; and c) cultivating the at least one mutant strain in a culture medium containing a digestible carbon source and a digestible nitrogen source under aerobic conditions.

In yet other aspects, screening the plurality of mutant strains comprises: i) culturing the plurality of mutant strains in medium containing gougerotin; ii) selecting mutant strains that survive in the medium containing gougerotin; and iii) quantifying the gougerotin produced by the selected mutant strains. The gougerotin concentration in the medium may be about 10 mg/mL, about 20 mg/mL, about 30 mg/mL, about 40 mg/mL, about 50 mg/mL, about 60 mg/mL, or about 70 mg/mL.

In some embodiments, the mutant strains are generated by genome shuffling. In certain aspects, the mutant strains are not generated by chemical mutagenesis with N-methyl-N′-nitro-N-nitrosoguanidine (NTG).

Also provided is a fermentation broth containing at least about 1 g/L gougerotin. In one embodiment the fermentation broth has not been subjected to any downstream processing. In a particular embodiment the fermentation broth is from a Streptomyces strain. Types of Streptomyces strains that are suitable for the invention are described in detail herein.

Also provided is a fermentation product of a gougerotin-producing Streptomyces strain, wherein the fermentation product comprises at least about 1 g/L gougerotin. In one embodiment the fermentation product is a fermentation broth. Also provided is a fermentation broth containing at least about 1 g/L, at least about 2 g/L, at least about 3 g/L, at least about 4 g/L, at least about 5 g/L, at least about 6 g/L, at least about 7 g/L or at least about 8 g/L gougerotin. In one embodiment, the fermentation broth contains gougerotin in a concentration of about 1 g/L to about 15 g/L. In one embodiment the gougerotin-producing Streptomyces strain is S. microflavus, S. griseus, S. anulatus, S. fimicarius, S. parvus, S. lavendulae, S. alboviridis, S. puniceus, or S. graminearus.

In yet another embodiment the gougerotin-producing Streptomyces strain is Streptomyces microflavus strain NRRL B-50550 or a phytophagous-miticidal and/or fungicidal mutant strain derived therefrom. In another it is Streptomyces puniceus strain A or a phytophagous-miticidal mutant strain derived therefrom. In yet another it is Streptomyces microflavus strain M. In yet another it is Streptomyces microflavus strain NRRL B-50954, Streptomyces microflavus strain NRRL B-50955, Streptomyces microflavus strain NRRL B-50956, Streptomyces microflavus strain NRRL B-50957, Streptomyces microflavus strain NRRL B-50958 or a mutant strain derived from any of the aforementioned strains.

The present invention also provides a method of producing a fermentation broth of a gougerotin producing Streptomyces strain, wherein the fermentation broth contains at least about 0.5 g/L gougerotin, the method comprising cultivating the Streptomyces strain in a culture medium containing a digestible carbon source and a digestible nitrogen source under aerobic conditions, wherein the culture medium contains an amino acid at a concentration effective to achieve a gougerotin concentration of at least 0.5 g/L. The present invention also provides a method of producing a fermentation broth of a gougerotin producing Streptomyces strain, wherein the fermentation broth contains at least about 1 g/L gougerotin, the method comprising cultivating the Streptomyces strain in a culture medium containing a digestible carbon source and a digestible nitrogen source under aerobic conditions, wherein the culture medium contains an amino acid at a concentration effective to achieve a gougerotin concentration of at least 1 g/L. The present invention also provides a method of treating a plant to control a plant disease or pest, wherein the method comprises applying the Streptomyces microflavus strain NRRL B-50550 or a phytophagous-miticidal and/or fungicidal mutant strain derived therefrom such as Streptomyces microflavus strain M, Streptomyces microflavus strain NRRL B-50954, Streptomyces microflavus strain NRRL B-50955, Streptomyces microflavus strain NRRL B-50956, Streptomyces microflavus strain NRRL B-50957, or Streptomyces microflavus strain NRRL B-50958, to the plant, to a part of the plant and/or to a locus of the plant. In one embodiment, a fermentation product of the strain or a fermentation product of a mutant derived therefrom is applied to the plant and/or to a locus of the plant.

The present invention also provides a method of treating a plant to control a plant disease or pest, wherein the method comprises applying a composition comprising a strain selected from the group consisting of Streptomyces microflavus strain NRRL B-50954, Streptomyces microflavus strain NRRL B-50955, Streptomyces microflavus strain NRRL B-50956, Streptomyces microflavus strain NRRL B-50957, Streptomyces microflavus strain NRRL B-50958, and mutants thereof having all the identifying characteristics of the respective strain, to the plant, to a part of the plant and/or to a locus of the plant. In one embodiment, a fermentation product of the strain or a fermentation product of a mutant derived therefrom is applied to the plant and/or to a locus of the plant. In another embodiment, the strain is Streptomyces microflavus strain NRRL B-50958 or a mutant thereof having all the identifying characteristics of the strain.

In certain aspects, the pest to be controlled is selected from a mite and Diabrotica spp. In other aspects plant disease is caused by a fungus. The plant disease may a leaf blotch disease or a leaf wilt disease such as those caused by Venturia sp. or Mycosphaerella sp. In yet other aspects, the plant disease is a mildew or a rust disease. The mildew may be powdery mildew or downy mildew.

In some embodiments, the powdery mildew is caused by a pathogen selected from the group consisting of Blumeria sp., Podosphaera sp., Sphaerotheca sp., and Uncinula sp. The pathogen may be Podosphaera xanthii. In certain aspects, the rust disease is selected from the group consisting of wheat leaf rust leaf rust caused by Puccinia triticina, leaf rust of barley caused by Puccinia hordei, leaf rust of rye caused by Puccinia recondita, brown leaf rust, crown rust, and stem rust.

In certain aspects, the present invention is directed to a method of treating a plant to control a mildew caused by Sphaerotheca sp. or Uncinula sp., a leaf blotch disease or a leaf wilt disease, wherein the method comprises applying a composition comprising Streptomyces microflavus strain NRRL B-50550 or a phytophagous-miticidal and/or fungicidal mutant strain derived therefrom, to the plant, to a part of the plant and/or to a locus of the plant. In some embodiments, the composition is a fermentation product of the strain. In other embodiments, the method comprises applying the composition to foliar plant parts. In yet other embodiments, the phytophagous-miticidal and/or fungicidal mutant strain is Streptomyces microflavus strain No. 091013-02.

In some aspects, the leaf blotch disease or a leaf wilt disease is caused by Venturia sp. or Mycosphaerella sp. In other aspects, the mildew is caused by a pathogen selected from the group consisting of Blumeria sp., Podosphaera sp., Sphaerotheca sp., and Uncinula sp. In one embodiment, the mildew is caused Sphaerotheca sp. In another embodiment, the mildew is caused Uncinula sp.

The invention also provides for a method of controlling phytophagous acari or insects comprising applying to a plant or to soil surrounding the plant a Streptomyces microflavus strain, including the Streptomyces microflavus strain NRRL B-50550 or a phytophagous-miticidal and/or fungicidal strain derived therefrom. In one embodiment, a fermentation product of the strain or a fermentation product of a mutant derived therefrom is applied to the plant and/or to a locus of the plant.

Also provided is a method of producing a fermentation broth of a gougerotin producing Streptomyces strain, wherein the fermentation broth contains at least about 1 g/L gougerotin, the method comprising cultivating the Streptomyces strain in a culture medium containing a digestible carbon source and a digestible nitrogen source under aerobic conditions, wherein the culture medium contains an amino acid at a concentration effective to achieve a gougerotin concentration of at least 1 g/L. In one embodiment, the Streptomyces strain is cultivated in the culture medium until the culture medium contains gougerotin in a concentration of at least about 2 g/L, of at least about 3 g/L, of at least about 4 g/L, of at least about 5 g/L, of at least about 6 g/L, of at least about 7 g/L or of at least about 8 g/L gougerotin. In another, the Streptomyces strain is cultivated in the culture medium until the culture medium contains gougerotin in a concentration ranging from about 1 g/L to about 15 g/L gougerotin.

In one embodiment of said method of producing a fermentation broth, the amino acid is selected from the group consisting of glycine, glutamic acid, glutamine, serine and mixtures thereof. In one instance, the culture medium contains the amino acid in an initial concentration of at least about 2 g/L. In a particular instance, the culture medium contains glycine at an initial concentration and/or glutamic acid at an initial concentration of about 5 g/L to about 15 g/L.

The culture medium, as described above, contains, in one embodiment, as carbon source a mixture of glucose and an oligosaccharide. In one instance, the oligosaccharide is maltodextrin or dextrin. In a particular instance, the initial maltodextrin concentration in the culture medium is about 50 g/L to about 100 g/L. In another, the initial maltodextrin concentration is about 60 g/L to about 80 g/L.

In one embodiment, the initial glucose concentration in the culture medium is about 20 g/L to 60 g/L or about 30 g/L to about 50 g/L.

In one embodiment, the culture medium contains calcium carbonate at an initial concentration of about 1 g/L to 3 g/L.

In one embodiment, the nitrogen source is at least partially selected from the group consisting of soy peptone, soy acid hydrolysate, soy flour hydrolysate, casein hydrolysate, yeast extract, and mixtures thereof.

Any of the gougerotin-producing Streptomyces strains described above may be used to practice this method.

Also provided is a method of enhancing gougerotin levels in a fermentation broth of a gougerotin-producing Streptomyces strain comprising cultivating the Streptomyces strain in a culture medium containing a digestible carbon source and a digestible nitrogen source under aerobic conditions, wherein the culture medium contains an amino acid at a concentration effective to achieve a gougerotin concentration that is at least two times greater than the gougerotin concentration achieved in a culture medium that contains less than about 1 g/L, about 2 g/L, about 3 g/L, about 4 g/L, about 5 g/L, about 6 g/L, about 7 g/L, about 8 g/L, about 9 g/L, or about 10 g/L, of one or more amino acids. In one embodiment the amino acid used in the culture medium is glutamic acid, serine and/or glycine. In one embodiment, the amino acid concentration in the culture medium used to obtain an enhanced level of gougerotin (i.e., the enhanced culture medium) is about 2 g/L to about 15 g/L and the gougerotin concentration achieved is at least two times that achieved in a starting culture medium, where, in one embodiment, the starting culture medium contains no more than about ½ the concentration of amino acids contained in the enhanced culture medium.

Any of the gougerotin-producing Streptomyces strains described above may be used to practice this method.

### BRIEF DESCRIPTION OF THE DRAWINGS

FIG. 1 shows the results of UV stability tests with bacterial candidate strains, when diluted fermentation products of the strains were sprayed on leafs of lima bean plants infested with two spotted spider mites. The columns in light gray (lower column for each strain) represent the antimiticidal activity without UV light irradiation, the columns in dark gray (upper column for each strain) represent the antimiticidal activity after irradiation with UV light for 24 hours. On the fifth day, plants were assessed for presence of mites and eggs on a scale of 1 to 4.

FIG. 2 shows the result of tests for translaminar activity of the bacterial candidate strains tested, when diluted fermentation products of the strains were sprayed on leafs of lima bean plants infested with two spotted spider mites. The columns in light gray (lower column for each strain) represent the translaminar (antimiticidal) activity, the columns in dark gray (upper column for each strain) represent the overall antimiticidal activity. On the sixth day, plants were assessed for presence of mites and eggs on a scale of 1 to 4.

FIG. 3 shows increased gougerotin production in mutants of Streptomyces microflavus strain NRRL B-50550 grown in 1 L shake flasks relative to the parent strain.

FIG. 4 shows increased gougerotin production in mutants of Streptomyces microflavus strain NRRL B-50550 grown in 5 L bioreactors relative to the parent strain.

FIG. 5 shows the chemical structure of gougerotin, as well as the serine, sugar, cytosine, and sarcosine subdomains thereof.

FIG. 6 shows gougerotin levels in genome-shuffled Streptomyces microflavus isolates.

### DETAILED DESCRIPTION OF INVENTION

All publications, patents and patent applications, including any drawings and appendices, herein are incorporated by reference to the same extent as if each individual publication or patent application was specifically and individually indicated to be incorporated by reference.

The following description includes information that may be useful in understanding the present invention. It is not an admission that any of the information provided herein is prior art or relevant to the presently claimed inventions, or that any publication specifically or implicitly referenced is prior art.

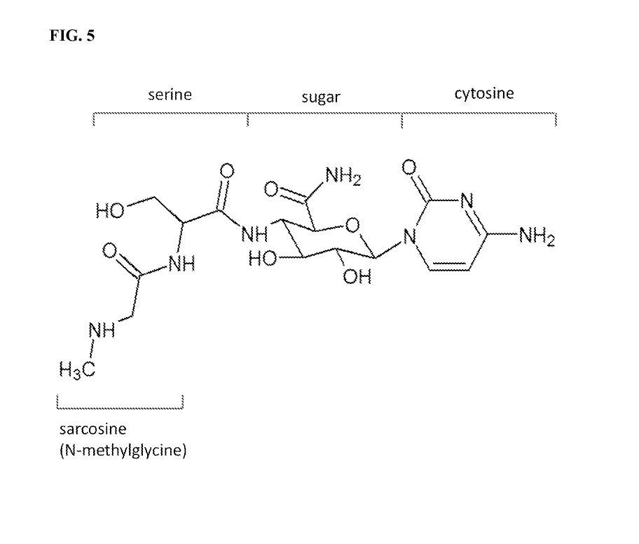

The present invention provides the Streptomyces microflavus strain NRRL B-50550 or a phytophagous-miticidal and/or fungicidal mutant strain derived therefrom. It has been found that the strain NRRL B-50550 has a variety of advantageous properties. Not only does the strain NRRL B-50550 (or its fermentation product) have acaricidal activity as such but, for example, also shows a high UV stability, a good translaminar activity, good ovicidal activity, long residual activity, drench activity as well as activity against a broad range of mites (see Example Section) and thus meets the requirements for an effective acaricide. In addition, the strain NRRL B-50550 (or its fermentation product) possesses both insecticidal activity and activity against various fungal phytopathogens such as leaf rust and mildew. This unique combination of activities makes the strain NRRL B-50550 a highly versatile candidate and renders the strain suitable to be broadly employed in methods of treating plants to control a plant disease and/or a plant pest. Such a broad range of activities and possible applications in agriculture has not yet been reported for known Streptomyces strains. In relation to a possible agricultural use, Streptomyces strains have been predominantly described in publications from the late 1960's and early 1970's. See, for example, the British Patent No. GB 1 507 193 that describes the Streptomyces rimofaciens strain No. B-98891, deposited as ATCC 31120, which produces the microbial compound B-98891. According to GB 1 507 193, filed March 1975, the microbial compound B-98891 is the active ingredient that provides antifungal activity of the Streptomyces rimofaciens strain No. B-98891 against powdery mildew. U.S. Pat. No. 3,849,398, filed Aug. 2, 1972, describes that the strain Streptomyces toyocaensis var. aspiculamyceticus produces the microbial compound aspiculamycin which is also known as gougerotin (see, Toru Ikeuchi et al., 25 J. Antibiotics 548 (September 1972). According to U.S. Pat. No. 3,849,398, gougerotin has parasiticidal action against parasites on animals, such as pin worm and the like, although gougerotin is said to show a weak antibacterial activity against gram-positive, gram-negative bacteria and tubercule bacillus. Similarly, Japanese Patent Application No. JP 53109998 (A), published 1978, reports the strain Streptomyces toyocaensis (LA-681) and its ability to produce gougerotin for use as miticide. However, it is to be noted that no miticidal fermentation product based on such Streptomcyes strains is commercially available. Thus, the Streptomyces microflavus strain NRRL B-50550 with its broad efficacy against acari (based on gougerotin production), fungi and insects and its favorable properties in terms of mode of action (e.g., translaminar activity and residual activity) represents a significant and unexpected advancement in terms of biological and advantageous properties which as such have not been reported for known Streptomyces strains. Applicant has solved the problem of producing a fermentation broth containing high concentrations of gougerotin, making feasible the ultimate use of the fermentation broth as a commercial pesticide or as a source of gougerotin for use as a commercial pesticide. Thus, this invention encompasses fermentation broths containing gougerotin at concentrations of at least about 0.5 g/L. In addition, this invention encompasses fermentation broths containing gougerotin at concentrations of at least about 1 g/L, at least about 2 g/L, at least about 3 g/L, at least about 4 g/L, at least about 5 g/L at least about 6 g/L, at least about 7 g/L or at least about 8 g/L or of at least about 1 mg/g, at least about 2 mg/g, at least about 3 mg/g, at least about 4 mg/g, at least about 5 mg/g, at least about 6 mg/g, at least about 7 mg/g or at least about 8 mg/g. In other embodiments the fermentation broth contains gougerotin in a concentration ranging from about 2 g/L to about 15 g/L, including in a concentration of about 3 g/L, of about 4 g/L, of about of about 5 g/L, of about 6 g/L, of about 7 g/L, of about 8 g/L, of about 9 g/L, of about of 10 g/L, of about 11 g/L, of about 12 g/L, of about 13 g/L, and of about 14 g/L or in a concentration ranging from about 2 mg/g to about 15 mg/g. In some embodiments the fermentation broths are from Streptomyces species. In specific embodiments, the fermentation broths are from Streptomyces microflavus. In still other specific embodiments, the fermentation broths are from Streptomyces microflavus NRRL-50550 or phytophagous-miticidal and/or fungicidal mutants derived therefrom. See structure of gougerotin below and in FIG. 5.

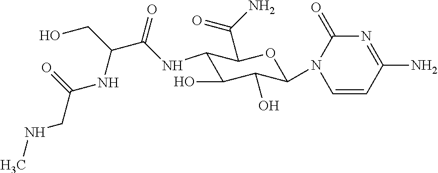 [SMILES: CNCC(=O)NC(CO)C(=O)NC1C(C(N)=O)OC(n2ccc(N)nc2=O)C(O)C1O]

The microorganisms and particular strains described herein, unless specifically noted otherwise, are all separated from nature (i.e., isolated) and grown under artificial conditions, such as in shake flask cultures or through scaled-up manufacturing processes, such as in bioreactors, as described herein. In one embodiment, a phytophagous-miticidal and/or fungicidal mutant strain of the Streptomyces microflavus strain NRRL B-50550 is provided. Streptomyces microflavus is a mesophilic, saprophytic bacterium belonging to the genus Streptomyces, found commonly in soil and decaying vegetation. NRRL B-50550 is a strain of Streptomyces microflavus that was isolated from soil in the continental United States of America. Streptomyces microflavus is an aerobic, Gram-positive, filamentous bacterium which produces well developed filamentous vegetative hyphae (˜1.0 μm wide and 10-100 μm long) and is capable of producing conidia-asexual spores. The hyphae consist of long, straight filaments, which bear beige, smooth spores at more or less regular intervals, arranged in whorls (verticils). Each branch of a verticil produces, at its apex, an umbel which carries from two to several chains of spores.

The term “mutant” refers to a genetic variant derived from Streptomyces microflavus strain NRRL B-50550, Streptomyces microflavus strain No. 091013-02, Streptomyces microflavus strain NRRL B-50954, Streptomyces microflavus strain NRRL B-50955, Streptomyces microflavus strain NRRL B-50956, Streptomyces microflavus strain NRRL B-50957, or Streptomyces microflavus strain NRRL B-50958. In one embodiment, the mutant has one or more or all the identifying (functional) characteristics of the respective Streptomyces microflavus strain. In a particular instance, the mutant or a fermentation product thereof controls (as an identifying functional characteristic) mites at least as well as the parent Streptomyces microflavus strain. In addition, the mutant or a fermentation product thereof may have one, two, three, four or all five of the following characteristics: translaminar activity in relation to the miticidal activity, residual activity in relation to the miticidal activity, ovicidal activity, insecticide activity, in particular against diabrotica, or activity against fungal phytopathogens, in particular against mildew and rust disease. Such mutants may be genetic variants having a genomic sequence that has greater than about 85%, greater than about 90%, greater than about 95%, greater than about 98%, or greater than about 99% sequence identity to the respective Streptomyces microflavus strain. Mutants may be obtained by treating a Streptomyces microflavus strain cells with chemicals or irradiation, by selecting spontaneous mutants from a population of the cells (such as phage resistant or antibiotic resistant mutants), by genome shuffling or cell fusion, and/or by other means well known to those practiced in the art.

Suitable chemicals for mutagenesis of Streptomcyes microflavus include hydroxylamine hydrochloride, methyl methanesulfonate (MMS), ethyl methanesulfonate (EMS), 4-nitroquinoline 1-oxide (NQO), mitomycin C or N-methyl-N′-nitro-N-nitrosoguanidine (NTG), to mention only a few (cf., for example, Stonesifer & Baltz, Proc. Natl. Acad. Sci. USA Vol. 82, pp. 1180-1183, February 1985). The mutagenesis of Streptomyces strains by, for example, NTG, using spore solutions of the respective Streptomcyes strain is well known to the person skilled in the art. See, for example Delic et al, Mutation Research/Fundamental and Molecular Mechanisms of Mutagenesis, Volume 9, Issue 2, February 1970, pages 167-182, or Chen et al., J Antibiot (Tokyo), 2001 November; 54(11), pages 967-972.). In more detail, Streptomyces microflavus can be subjected to mutation by NTG using the protocol described in Kieser, T., et al., 2000, supra. Practical Streptomyces Genetics, Ch. 5 John Innes Centre, Norwich Research Park, England (2000), pp. 99-107. Mutagenesis of spores of Streptomyces microflavus by ultraviolet light (UV) can be carried out using standard protocols. For example, a spore suspension of the Streptomyces strain (freshly prepared or frozen in 20% glycerol) can be suspended in a medium that does not absorb UV light at a wave length of 254 nm (for example, water or 20% glycerol are suitable). The spore suspension is then placed in a glass Petri dish and irradiated with a low pressure mercury vapour lamp that emits most of its energy at 254 nm with constant agitation for an appropriate time at 30° C. (the most appropriate time of irradiation can be determined by first plotting a dose-survival curve). Slants or plates of non-selective medium can, for example, then be inoculated with the dense irradiated spore suspension and the so obtained mutant strains can be assessed for their properties as explained in the following. See Kieser, T., et al., 2000, supra.

Alternatively mutants can be generated through the process of genome shuffling (also known as cell fusion) using protocols similar to those described in Kieser, T., et al., 2000, supra. Practical Streptomyces Genetics, Ch. 5 John Innes Centre, Norwich Research Park, England (2000), pp. 56-58, 156-160, 408, 412, 415. Genome shuffling utilizes a pool (typically between 5 and 10 individual mutants per round of shuffle) of previously generated and characterized mutants from which protoplasts (intact individual bacterial spheres with the cell wall components removed through enzymatic digestion or mechanical disruption) are generated and combined in a suitable environment (polyethylene glycol or similar solution previously determined experimentally as ideal for a specific bacterial strain) for the random exchange of their genetic material. Variants isolated from genome shuffling screens are evaluated for their ability to produce miticidal compounds and/or fungicidal compounds compared with the mutants from which they were derived.

The mutant strain can be any mutant strain that has one or more or all the identifying characteristics of Streptomyces microflavus strain NRRL B-50550, Streptomyces microflavus strain No. 091013-02, Streptomyces microflavus strain NRRL B-50954, Streptomyces microflavus strain NRRL B-50955, Streptomyces microflavus strain NRRL B-50956, Streptomyces microflavus strain NRRL B-50957, or Streptomyces microflavus strain NRRL B-50958 and in particular miticidal and/or fungicidal activity that is comparable or better than that of the respective Streptomyces microflavus strain. The miticidal activity can, for example, be determined against two-spotted spider mites (“TSSM”) as explained in Example 2 herein, meaning culture stocks of the mutant strain can be grown in 1 L shake flasks in Media 1 or Media 2 of Example 2 at 20-30° C. for 3-5 days, and the diluted fermentation product can then be applied on top and bottom of lima bean leaves of two plants, after which treatment, plants can be infested on the same day with 50-100 TSSM and left in the greenhouse for five days.

Example 15 provides a specific example of a method for generating mutants of Streptomyces microflavus strain NRRL B-50550. One mutant generated by this method is Streptomyces microflavus strain M, which is described more fully in the examples. Mutants derived, in turn, from Streptomyces microflavus strain M are also described in the examples, including Streptomyces microflavus strain NRRL B-50954, Streptomyces microflavus strain NRRL B-50955, Streptomyces microflavus strain NRRL B-50956, Streptomyces microflavus strain NRRL B-50957, and Streptomyces microflavus strain NRRL B-50958.

In one aspect of the invention, the Streptomyces microflavus strain NRRL B-50550 or a phytophagous-miticidal and/or fungicidal mutant strain thereof has translaminar activity. The term “translaminar activity” is used herein in its regular meaning in the art and thus by “translaminar activity” is meant the ability of a compound or composition (here a composition such as a fermentation product containing the Streptomyces microflavus strain NRRL B-50550 or a mutant strain thereof) of moving through the leaf tissue of the plant to be treated. A translaminar compound/composition penetrates leaf tissues and forms a reservoir of active ingredient within the leaf. This translaminar activity therefore also provides residual activity against foliar-feeding insects and mites. Because the composition (or its one or more active ingredients) can move through leaves, thorough spray coverage is less critical to control acari such as mites, which normally feed on leaf undersides. The translaminar activity of a mutant strain alone or in comparison to Streptomyces microflavus NRRL B-50550 can, for example, be determined against two-spotted spider mites (“TSSM”) as explained in Example 6 herein. Translaminar activity can still be observed after several days (e.g., about 5 days) under the conditions of Example 6. In one aspect of the invention, translaminar activity can be observed (is present) at least about 1, 2, 3, 4, 5, 6, 7, 8, 9, and/or 10 days after treatment.

In another aspect of the invention, the Streptomyces microflavus strain NRRL B-50550 or a phytophagous-miticidal and/or fungicidal mutant strain thereof has residual activity. The term “residual activity” is used herein in its regular meaning in the art and thus by “residual activity” is meant the ability of a compound or composition (here a composition such as a fermentation product containing the Streptomyces microflavus strain NRRL B-50550 or a mutant strain thereof) to remain effective (i.e., cause greater mortality of mites or cause a reduction in the total number of mites, versus conditions where the compound or composition was not applied) for an extended period of time after it is applied. The length of time may depend on the formulation (dust, liquid, etc.), the type of plant or location and the condition of the plant surface or soil surface (wet, dry, etc.) to which a composition containing Streptomyces microflavus strain NRRL B-50550 or a mutant strain thereof is applied. The residual activity of a mutant strain alone or in comparison to Streptomyces microflavus NRRL B-50550 can, for example, be determined against two-spotted spider mites (“TSSM”) as explained in Example 2 or 7 herein and means, in relation to the miticidal effect, that an antimiticidal effect can still be observed after several days (e.g., about 12 days) under the conditions of Example 5. In one aspect of the invention, residual activity can be observed (is present) at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and/or 40 days after treatment.

In another aspect of the invention, the Streptomyces microflavus strain NRRL B-50550 or a phytophagous-miticidal and/or fungicidal mutant strain thereof has ovicidal activity. The term “ovicidal activity” is used herein in its regular meaning in the art to mean “the ability of causing destruction or death of an ovum” and is used herein in relation to eggs of acari such as mites. The ovicidal activity of a mutant strain of Streptomyces microflavus NRRL B-50550 alone or in comparison to Streptomyces microflavus NRRL B-50550 can be determined using the method as described in Example 7. Ovicidal activity can still be observed after several days (e.g., about 5 days) under the conditions of Example 7. In one aspect of the invention, ovicidal activity can be observed (is present) at least about 1, 2, 3, 4, 5, 6, 7, 8, 9, and/or 10 days after treatment.

In another aspect of the invention, the Streptomyces microflavus strain NRRL B-50550 or a phytophagous-miticidal and/or fungicidal mutant strain thereof may have drench activity. The term “drench activity” is used herein in its regular meaning in the art to mean pesticidal activity that travels from soil or other growth media upward through the plant via the xylem. The drench activity of a mutant strain of Streptomyces microflavus NRRL B-50550 alone or in comparison to Streptomyces microflavus NRRL B-5055 can be determined using the method as described in Example 8. In one aspect of the invention, drench activity can still be observed (is present) after several days (e.g., about 2, 3, 4, 5, 6, 7, 8, 9, 10, or 11 days) under the conditions of Example 8.

In another aspect of the invention, the Streptomyces microflavus strain NRRL B-50550 or a phytophagous-miticidal and/or fungicidal mutant strain thereof has miticidal activity against a variety of mite species, including, as illustrated in the Examples, but not limited to, activity against two-spotted spider mites, activity against citrus rust mites (Phyllocoptruta oleivora), eriophyid (russet) mites and broad mites.

The Streptomyces microflavus strain NRRL B-50550 or a phytophagous-miticidal and/or fungicidal mutant strain thereof may thus have activity against a mite that is selected from the group consisting of clover mite, brown mite, hazelnut spider mite, asparagus spider mite, brown wheat mite, legume mite, oxalis mite, boxwood mite, Texas citrus mite, Oriental red mite, citrus red mite, European red mite, yellow spider mite, fig spider mite, Lewis spider mite, six-spotted spider mite, Willamette mite, Yuma spider mite, web-spinning mite, pineapple mite, citrus green mite, honey-locust spider mite, tea red spider mite, southern red mite, avocado brown mite, spruce spider mite, avocado red mite, Banks grass mite, carmine spider mite, desert spider mite, vegetable spider mite, tumid spider mite, strawberry spider mite, two-spotted spider mite, McDaniel mite, Pacific spider mite, hawthorn spider mite, four-spotted spider mite, Schoenei spider mite, Chilean false spider mite, citrus flat mite, privet mite, flat scarlet mite, white-tailed mite, pineapple tarsonemid mite, West Indian sugar cane mite, bulb scale mite, cyclamen mite, broad mite, winter grain mite, red-legged earth mite, filbert big-bud mite, grape erineum mite, pear blister leaf mite, apple leaf edgeroller mite, peach mosaic vector mite, alder bead gall mite, Perian walnut leaf gall mite, pecan leaf edgeroll mite, fig bud mite, olive bud mite, citrus bud mite, litchi erineum mite, wheat curl mite, coconut flower and nut mite, sugar cane blister mite, buffalo grass mite, bermuda grass mite, carrot bud mite, sweet potato leaf gall mite, pomegranate leaf curl mite, ash sprangle gall mite, maple bladder gall mite, alder erineum mite, redberry mite, cotton blister mite, blueberry bud mite, pink tea rust mite, ribbed tea mite, grey citrus mite, sweet potato rust mite, horse chestnut rust mite, citrus rust mite, apple rust mite, grape rust mite, pear rust mite, flat needle sheath pine mite, wild rose bud and fruit mite, dryberry mite, mango rust mite, azalea rust mite, plum rust mite, peach silver mite, apple rust mite, tomato russet mite, pink citrus rust mite, cereal rust mite, rice rust mite and combinations thereof. In addition, the Streptomyces microflavus strain NRRL B-50550 or a phytophagous-miticidal and/or fungicidal mutant strain thereof has activity against mites that are resistant to other mite control agents. In one embodiment, the strain has activity against abamectin-resistant mites.

In another aspect of the invention, the Streptomyces microflavus strain NRRL B-50550 or a phytophagous-miticidal and/or fungicidal mutant strain thereof may have also insecticide activity. The target insect may be a diabrotica. The Diabrotica may be banded cucumber beetle (Diabrotica balteata), Western spotted cucumber beetle (Diabrotica undecimpunctata undecimpunctata), or a corn rootworm such as Northern corn rootworm (Diabrotica barberi), Southern corn rootworm (Diabrotica undecimpunctata howardi), Western cucumber beetle (Diabrotica undecimpunctata tenella), Western corn rootworm (Diabrotica virgifera virgifera), Mexican corn rootworm (Diabrotica virgifera zeae) and combinations of such Diabrotica. The insecticidal activity of a mutant strain of Streptomyces microflavus NRRL B-50550 alone or in comparison to Streptomyces microflavus NRRL B-50550 can be determined against corn rootworm, using the method as described in Example 10.

In another aspect of the invention, the Streptomyces microflavus strain NRRL B-50550 or a phytophagous-miticidal and/or fungicidal mutant strain thereof has fungicide activity, meaning activity against a plant disease that is caused by a fungus. The plant disease may be mildew or a rust disease. Examples of mildew that can be treated with the Streptomyces microflavus strain NRRL B-50550 or a phytophagous-miticidal and/or fungicidal mutant strain thereof include, but are not limited to, powdery mildew, such as cucumber powdery mildew caused by Sphaerotheca fuliginea, or downy mildew, such as brassica downy mildew, caused by Peronospora parasitica. Examples of a rust disease that may be treated with Streptomyces microflavus strain NRRL B-50550 or a phytophagous-miticidal and/or fungicidal mutant strain thereof include, but are not limited to, wheat leaf rust caused by Puccinia triticina (also known as P. recondita), wheat stem rust caused by Puccinia grammis, wheat stripe rust caused by Puccinia striiformis, leaf rust of barley caused by Puccinia hordei, leaf rust of rye caused by Puccinia recondita, brown leaf rust, crown rust, and stem rust. The fungicidal activity of a mutant strain of Streptomyces microflavus NRRL B-50550 alone or in comparison to Streptomyces microflavus NRRL B-50550 can be determined against cucumber powdery mildew using the method as described in Example 9. Fungicidal activity can still be observed after several days (e.g., about 7 days) under the conditions of Example 9. In one aspect of the invention, fungicidal activity can be observed (is present) about 1, 2, 3, 4, 5, 6, 7, 8, 9, 10, 11, 12, 13, 14, and/or 15 days after treatment.

The inventive compositions have potent microbicidal activity and can be used for control of unwanted microorganisms, such as fungi and bacteria, in crop protection and in the protection of materials.

The invention also relates to a method for controlling unwanted microorganisms, characterized in that the inventive compositions are applied to the phytopathogenic fungi, phytopathogenic bacteria and/or their habitat.

Fungicides can be used in crop protection for control of phytopathogenic fungi. They are characterized by an outstanding efficacy against a broad spectrum of phytopathogenic fungi, including soilborne pathogens, which are in particular members of the classes Plasmodiophoromycetes, Peronosporomycetes (Syn. Oomycetes), Chytridiomycetes, Zygomycetes, Ascomycetes, Basidiomycetes and Deuteromycetes (Syn. Fungi imperfecti). Some fungicides are systemically active and can be used in plant protection as foliar, seed dressing or soil fungicide. Furthermore, they are suitable for combating fungi, which inter alia infest wood or roots of plant.

Bactericides can be used in crop protection for control of Pseudomonadaceae, Rhizobiaceae, Enterobacteriaceae, Corynebacteriaceae and Streptomycetaceae.

Non-limiting examples of pathogens of fungal diseases which can be treated in accordance with the invention include:

diseases caused by powdery mildew pathogens, for example Blumeria species, for example Blumeria graminis; Podosphaera species, for example Podosphaera leucotricha; Sphaerotheca species, for example Sphaerotheca fuliginea; Uncinula species, for example Uncinula necator;

diseases caused by rust disease pathogens, for example Gymnosporangium species, for example Gymnosporangium sabinae; Hemileia species, for example Hemileia vastatrix; Phakopsora species, for example Phakopsora pachyrhizi and Phakopsora meibomiae; Puccinia species, for example Puccinia recondite, P. triticina, P. graminis or P. striiformis; Uromyces species, for example Uromyces appendiculatus;

diseases caused by pathogens from the group of the Oomycetes, for example Albugo species, for example Algubo candida; Bremia species, for example Bremia lactucae; Peronospora species, for example Peronospora pisi or P. brassicae; Phytophthora species, for example Phytophthora infestans; Plasmopara species, for example Plasmopara viticola; Pseudoperonospora species, for example Pseudoperonospora humuli or Pseudoperonospora cubensis; Pythium species, for example Pythium ultimum;

leaf blotch diseases and leaf wilt diseases caused, for example, by Alternaria species, for example Alternaria solani; Cercospora species, for example Cercospora beticola; Cladiosporium species, for example Cladiosporium cucumerinum; Cochliobolus species, for example Cochliobolus sativus (conidia form: Drechslera, Syn: Helminthosporium), Cochliobolus miyabeanus; Colletotrichum species, for example Colletotrichum lindemuthanium; Cycloconium species, for example Cycloconium oleaginum; Diaporthe species, for example Diaporthe citri; Elsinoe species, for example Elsinoe fawcettii; Gloeosporium species, for example Gloeosporium laeticolor; Glomerella species, for example Glomerella cingulata; Guignardia species, for example Guignardia bidwelli; Leptosphaeria species, for example Leptosphaeria maculans, Leptosphaeria nodorum; Magnaporthe species, for example Magnaporthe grisea; Marssonia species, for example Marssonia coronaria; Microdochium species, for example Microdochium nivale; Mycosphaerella species, for example Mycosphaerella graminicola, M. arachidicola and M. fijiensis; Phaeosphaeria species, for example Phaeosphaeria nodorum; Pyrenophora species, for example Pyrenophora teres, Pyrenophora tritici repentis; Ramularia species, for example Ramularia collo-cygni, Ramularia areola; Rhynchosporium species, for example Rhynchosporium secalis; Septoria species, for example Septoria apii, Septoria lycopersii; Typhula species, for example Typhula incarnata; Venturia species, for example Venturia inaequalis;

root and stem diseases caused, for example, by Corticium species, for example Corticium graminearum; Fusarium species, for example Fusarium oxysporum; Gaeumannomyces species, for example Gaeumannomyces graminis; Rhizoctonia species, such as, for example Rhizoctonia solani; Sarocladium diseases caused for example by Sarocladium oryzae; Sclerotium diseases caused for example by Sclerotium oryzae; Tapesia species, for example Tapesia acuformis; Thielaviopsis species, for example Thielaviopsis basicola;

ear and panicle diseases (including corn cobs) caused, for example, by Alternaria species, for example Alternaria spp.; Aspergillus species, for example Aspergillus flavus; Cladosporium species, for example Cladosporium cladosporioides; Claviceps species, for example Claviceps purpurea; Fusarium species, for example Fusarium culmorum; Gibberella species, for example Gibberella zeae; Monographella species, for example Monographella nivalis; Septoria species, for example Septoria nodorum;

diseases caused by smut fungi, for example Sphacelotheca species, for example Sphacelotheca reiliana; Tilletia species, for example Tilletia caries, T. controversa; Urocystis species, for example Urocystis occulta; Ustilago species, for example Ustilago nuda, U. nuda tritici;

fruit rot caused, for example, by Aspergillus species, for example Aspergillus flavus; Botrytis species, for example Botrytis cinerea; Penicillium species, for example Penicillium expansum and P. purpurogenum; Sclerotinia species, for example Sclerotinia sclerotiorum; Verticilium species, for example Verticilium alboatrum;

seed and soilborne decay, mould, wilt, rot and damping-off diseases caused, for example, by Alternaria species, caused for example by Alternaria brassicicola; Aphanomyces species, caused for example by Aphanomyces euteiches; Ascochyta species, caused for example by Ascochyta lentis; Aspergillus species, caused for example by Aspergillus flavus; Cladosporium species, caused for example by Cladosporium herbarum; Cochliobolus species, caused for example by Cochliobolus sativus; (Conidiaform: Drechslera, Bipolaris Syn: Helminthosporium); Colletotrichum species, caused for example by Colletotrichum coccodes; Fusarium species, caused for example by Fusarium culmorum; Gibberella species, caused for example by Gibberella zeae; Macrophomina species, caused for example by Macrophomina phaseolina; Monographella species, caused for example by Monographella nivalis; Penicillium species, caused for example by Penicillium expansum; Phoma species, caused for example by Phoma lingam; Phomopsis species, caused for example by Phomopsis sojae; Phytophthora species, caused for example by Phytophthora cactorum; Pyrenophora species, caused for example by Pyrenophora graminea; Pyricularia species, caused for example by Pyricularia oryzae; Pythium species, caused for example by Pythium ultimum; Rhizoctonia species, caused for example by Rhizoctonia solani; Rhizopus species, caused for example by Rhizopus oryzae; Sclerotium species, caused for example by Sclerotium rolfsii; Septoria species, caused for example by Septoria nodorum; Typhula species, caused for example by Typhula incarnata; Verticillium species, caused for example by Verticillium dahliae;

cancers, galls and witches' broom caused, for example, by Nectria species, for example Nectria galligena;

wilt diseases caused, for example, by Monilinia species, for example Monilinia laxa;

leaf blister or leaf curl diseases caused, for example, by Exobasidium species, for example Exobasidium vexans;

Taphrina species, for example Taphrina deformans;

decline diseases of wooden plants caused, for example, by Esca disease, caused for example by Phaemoniella clamydospora, Phaeoacremonium aleophilum and Fomitiporia mediterranea; Eutypa dyeback, caused for example by Eutypa lata; Ganoderma diseases caused for example by Ganoderma boninense; Rigidoporus diseases caused for example by Rigidoporus lignosus;

diseases of flowers and seeds caused, for example, by Botrytis species, for example Botrytis cinerea;

diseases of plant tubers caused, for example, by Rhizoctonia species, for example Rhizoctonia solani; Helminthosporium species, for example Helminthosporium solani;

Club root caused, for example, by Plasmodiophora species, for example Plamodiophora brassicae;

diseases caused by bacterial pathogens, for example Xanthomonas species, for example Xanthomonas campestris pv. oryzae; Pseudomonas species, for example Pseudomonas syringae pv. lachrymans; Erwinia species, for example Erwinia amylovora.

The following diseases of soya beans can be controlled with preference:

Fungal diseases on leaves, stems, pods and seeds caused, for example, by Alternaria leaf spot (Alternaria spec. atrans tenuissima), Anthracnose (Colletotrichum gloeosporoides dematium var. truncatum), brown spot (Septoria glycines), cercospora leaf spot and blight (Cercospora kikuchii), choanephora leaf blight (Choanephora infiindibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi, Phakopsora meibomiae), scab (Sphaceloma glycines), stemphylium leaf blight (Stemphylium botryosum), target spot (Corynespora cassiicola).

Fungal diseases on roots and the stem base caused, for example, by black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

The inventive fungicidal compositions can be used for curative or protective/preventive control of phytopathogenic fungi. The invention therefore also relates to curative and protective methods for controlling phytopathogenic fungi by the use of the inventive compositions, which are applied to the seed, the plant or plant parts, the fruit or the soil in which the plants grow.

The present invention also provides a Streptomyces puniceus strain A or a phytophagous-miticidal and/or fungicidal mutant strain derived therefrom. Streptomyces puniceus is a member of the S. griseus clade of the Streptomyces bacterium. S. puniceus is an aerobic, gram positive, filamentous bacteria. It produces moderately long mature spore chains with 10 to more than 50 spores per chain. The spore texture is smooth and colony is yellowish to reddish in color when growing on oatmeal based agar. Streptomyces puniceus strain A was isolated from a soil sample collected in the continental United States of America. A fermentation product of strain A has miticidal properties, as described in Example 18. In one embodiment, a phytophagous-miticidal and/or fungicidal mutant strain of the Streptomyces puniceus strain A is provided. The term “mutant” refers to a genetic variant derived from Streptomyces puniceus strain A. In one embodiment, the mutant has one or more or all the identifying (functional) characteristics of Streptomyces puniceus strain A. In a particular instance, the mutant or a fermentation product thereof controls (as an identifying functional characteristic) mites at least as well as the parent Streptomyces puniceus strain A. Such mutants may be genetic variants having a genomic sequence that has greater than about 85%, greater than about 90%, greater than about 95%, greater than about 98%, or greater than about 99% sequence identity to Streptomyces puniceus strain A. Mutants may be obtained by treating Streptomyces puniceus strain A cells with chemicals or irradiation or by selecting spontaneous mutants from a population of A cells (such as phage resistant or antibiotic resistant mutants) or by other means well known to those practiced in the art, including those means described above and in Example 15 in reference to Streptomyces microflavus NRRL B-50550. Streptomyces puniceus strain A contains a gougerotin gene cluster that encodes proteins GouB-GouM and is anticipated to contain GouA. Proteins GouB-GouM of Streptomyces puniceus strain A have at least 90% sequence identity to the orthologous proteins from Streptomyces microflavus NRRL B-50550.

The present invention also encompasses methods of treating a plant to control plant pests and diseases by administering to a plant or a plant part, such as a leaf, stem, flowers, fruit, root, or seed or by applying to a locus on which plant or plant parts grow, such as soil, one or more of a gougerotin containing fermentation broth of Streptomcyes, the Streptomyces microflavus strain NRRL B-50550 or a phytophagous-miticidal or fungicidal mutant strain thereof or cell-free preparations thereof or metabolites thereof or the Streptomyces puniceus strain A or a phytophagous-miticidal and/or fungicidal mutant strain thereof or cell-free preparations thereof or metabolites thereof. Additional gougerotin-producing strains that are suitable for the methods and fermentation products of the present invention are described herein.

As used herein, the term “plant” refers to any living organism belonging to the kingdom Plantae (i.e., any genus/species in the Plant Kingdom). This includes familiar organisms such as but not limited to trees, herbs, bushes, grasses, vines, ferns, mosses and green algae. The term refers to both monocotyledonous plants, also called monocots, and dicotyledonous plants, also called dicots. The plant is in some embodiments of economic importance. In some embodiments the plant is a human-grown plant, for instance a cultivated plant, which may be an agricultural, a silvicultural or a horticultural plant. Examples of particular plants include but are not limited to corn, potatoes, roses, apple trees, sunflowers, wheat, rice, bananas, tomatoes, opo, pumpkins, squash, beans (e.g., lima beans), lettuce, cabbage, oak trees, guzmania, geraniums, hibiscus, clematis, poinsettias, sugarcane, taro, duck weed, pine trees, Kentucky blue grass, zoysia, coconut trees, brassica leafy vegetables (e.g., broccoli, broccoli raab, Brussels sprouts, cabbage, Chinese cabbage (Bok Choy and Napa), cauliflower, cavalo, collards, kale, kohlrabi, mustard greens, rape greens, and other brassica leafy vegetable crops), bulb vegetables (e.g., garlic, leek, onion (dry bulb, green, and Welch), shallot, and other bulb vegetable crops), citrus fruits (e.g., grapefruit, lemon, lime, orange, tangerine, citrus hybrids, pummelo, and other citrus fruit crops), cucurbit vegetables (e.g., cucumber, citron melon, edible gourds, gherkin, muskmelons (including hybrids and/or cultivars of cucumis melons), watermelon, cantaloupe, and other cucurbit vegetable crops), fruiting vegetables (including eggplant, ground cherry, pepino, pepper, tomato, tomatillo, and other fruiting vegetable crops), grape, leafy vegetables (e.g., romaine), root/tuber and corm vegetables (e.g., potato), lentils, alfalfa sprouts, clover and tree nuts (almond, pecan, pistachio, and walnut), berries (e.g., tomatoes, barberries, currants, elderberries, gooseberries, honeysuckles, mayapples, nannyberries, Oregon-grapes, see-buckthorns, hackberries, bearberries, lingonberries, strawberries, sea grapes, blackberries, cloudberries, loganberries, raspberries, salmonberries, thimbleberries, and wineberries), cereal crops (e.g., corn, rice, wheat, barley, sorghum, millets, oats, ryes, triticales, buckwheats, fonio, and quinoa), pome fruit (e.g., apples, pears), stone fruits (e.g., coffees, jujubes, mangos, olives, coconuts, oil palms, pistachios, almonds, apricots, cherries, damsons, nectarines, peaches and plums), vine (e.g., table grapes, wine grapes), fibber crops (e.g., hemp, cotton), ornamentals, to name a few. The plant may, in some embodiments, be a household/domestic plant, a greenhouse plant, an agricultural plant, or a horticultural plant. As already indicated above, in some embodiments the plant may a hardwood such as one of acacia, eucalyptus, hornbeam, beech, mahogany, walnut, oak, ash, willow, hickory, birch, chestnut, poplar, alder, maple, sycamore, ginkgo, a palm tree and sweet gum. In some embodiments the plant may be a conifer such as a cypress, a Douglas fir, a fir, a sequoia, a hemlock, a cedar, a juniper, a larch, a pine, a redwood, spruce and yew. In some embodiments the plant may be a fruit bearing woody plant such as apple, plum, pear, banana, orange, kiwi, lemon, cherry, grapevine, papaya, peanut, and fig. In some embodiments the plant may be a woody plant such as cotton, bamboo and a rubber plant. The plant may in some embodiments be an agricultural, a silvicultural and/or an ornamental plant, i.e., a plant which is commonly used in gardening, e.g., in parks, gardens and on balconies. Examples are turf, geranium, pelargonia, petunia, begonia, and fuchsia, to name just a few among the vast number of ornamentals. The term “plant” is also intended to include any plant propagules.

The term “plant” generally includes a plant that has been modified by one or more of breeding, mutagenesis and genetic engineering. Genetic engineering refers to the use of recombinant DNA techniques. Recombinant DNA techniques allow modifications which cannot readily be obtained by cross breeding under natural circumstances, mutations or natural recombination. In some embodiments a plant obtained by genetic engineering may be a transgenic plant.

As used herein, the term “plant part” refers to any part of a plant including but not limited to the shoot, root, stem, seeds, stipules, leaves, petals, flowers, ovules, bracts, branches, petioles, internodes, bark, wood, tubers, pubescence, tillers, rhizomes, fronds, blades, pollen, stamen, microspores, fruit and seed. The two main parts of plants grown in typical media employed in the art, such as soil, are often referred to as the “above-ground” part, also often referred to as the “shoots”, and the “below-ground” part, also often referred to as the “roots”.

In a method according to the invention a composition containing Streptomyces microflavus NRRL B-50550 or a phytophagous-miticidal and/or fungicidal mutant strain thereof can be applied to any plant or any part of any plant grown in any type of media used to grow plants (e.g., soil, vermiculite, shredded cardboard, and water) or applied to plants or the parts of plants grown aerially, such as orchids or staghorn ferns. The composition may for instance be applied by spraying, atomizing, vaporizing, scattering, dusting, watering, squirting, sprinkling, pouring or fumigating. As already indicated above, application may be carried out at any desired location where the plant of interest is positioned, such as agricultural, horticultural, forest, plantation, orchard, nursery, organically grown crops, turfgrass and urban environments.

Compositions of the present invention can be obtained by culturing Streptomyces microflavus NRRL B-50550 or mutants derived from it using conventional large-scale microbial fermentation processes, such as submerged fermentation, solid state fermentation or liquid surface culture, including the methods described, for example, in U.S. Pat. No. 3,849,398; British Patent No. GB 1 507 193; Toshiko Kanzaki et al., Journal of Antibiotics, Ser. A, Vol. 15, No. 2, June 1961, pages 93 to 97; or Toru Ikeuchi et al., Journal of Antibiotics, (September 1972), pages 548 to 550. Fermentation is configured to obtain high levels of live biomass, including spores, and desirable secondary metabolites in the fermentation vessels. Specific fermentation methods that are suitable for the strain of the present invention to achieve high levels of sporulation, cfu (colony forming units), and secondary metabolites are described in the Examples section.

The bacterial cells, spores and metabolites in culture broth resulting from fermentation (the “whole broth” or “fermentation broth”) may be used directly or concentrated by conventional industrial methods, such as centrifugation, filtration, and evaporation, or processed into dry powder and granules by spray drying, drum drying and freeze drying, for example.

The terms “whole broth” and “fermentation broth,” as used herein, refer to the culture broth resulting from fermentation (including the production of a culture broth that contains gougerotin in a concentration of at least about 1 g/L) before any downstream treatment. The whole broth encompasses the microorganism (e.g., Streptomyces microflavus NRRL B-50550 or a phytophagous-miticidal and/or fungicidal mutant strain thereof) and its component parts, unused raw substrates, and metabolites produced by the microorganism during fermentation. The term “broth concentrate,” as used herein, refers to whole broth (fermentation broth) that has been concentrated by conventional industrial methods, as described above, but remains in liquid form. The term “fermentation solid,” as used herein, refers to dried fermentation broth. The term “fermentation product,” as used herein, refers to whole broth, broth concentrate and/or fermentation solids. Compositions of the present invention include fermentation products. In some embodiments, the concentrated fermentation broth is washed, for example, via a diafiltration process, to remove residual fermentation broth and metabolites.

In one embodiment, the fermentation broth contains at least about 1×10⁵ colony forming units (CFU) of the microorganism (e.g., Streptomyces microflavus NRRL B-50550 or a phytophagous-miticidal and/or fungicidal mutant strain thereof)/mL broth. In another embodiment, the fermentation broth contains at least about 1×10⁶ colony forming units (CFU) of the microorganism (e.g., Streptomyces microflavus NRRL B-50550 or a phytophagous-miticidal mutant strain thereof)/mL broth. In yet another embodiment, the fermentation broth contains at least about 1×10⁷ colony forming units (CFU) of the microorganism (e.g., Streptomyces microflavus NRRL B-50550 or a phytophagous-miticidal and/or fungicidal mutant strain thereof)/mL broth. In another embodiment, the fermentation broth contains at least about 1×10⁸ colony forming units (CFU) of the microorganism (e.g., Streptomyces microflavus NRRL B-50550 or a phytophagous-miticidal and/or fungicidal mutant strain thereof)/mL broth. In another embodiment, the fermentation broth contains at least about 1×10⁹ colony forming units (CFU) of the microorganism (e.g., Streptomyces microflavus NRRL B-50550 or a phytophagous-miticidal mutant strain thereof)/mL broth. In another embodiment, the fermentation broth contains at least about 1×10¹⁰ colony forming units (CFU) of the microorganism (e.g., Streptomyces microflavus NRRL B-50550 or a phytophagous-miticidal and/or fungicidal mutant strain thereof)/mL broth. In another embodiment, the fermentation broth contains at least about 1×10¹¹ colony forming units (CFU) of the microorganism (e.g., Streptomyces microflavus NRRL B-50550 or a phytophagous-miticidal and/or fungicidal mutant strain thereof)/mL broth. One of skill in the art will understand that the concentrations described above relate to CFU measured shortly after completion of fermentation but that CFU levels will decline over time, depending on storage conditions. CFU levels of unformulated fermentation products of the microorganisms described herein are stable when the products are maintained in cold storage (e.g., about 4° C.) but decline at room temperature.

In one embodiment, the fermentation broth or broth concentrate can be formulated into liquid suspension, liquid concentrate, emulsion concentrate, or wettable powder with the addition of stabilization agents, preservatives, adjuvants, and/or colorants.

In another embodiment, the fermentation broth or broth concentrate can be dried with or without the addition of carriers, inerts, or additives using conventional drying processes or methods such as spray drying, freeze drying, tray drying, fluidized-bed drying, drum drying, or evaporation.

In some embodiments, the fermentation broth, broth concentrate or fermentation solid is treated in order to kill the microorganism, resulting in a fermentation product that consists of the killed microbe, its metabolites and residual fermentation media. Suitable treatments to accomplish this are known to those of skill in the art and include heat treatments.

In embodiments in which the fermentation broth or broth concentrate is freeze dried, one gallon of fermentation broth yields about 0.2 lb to about 1 lb freeze dried powder. In a particular instance, one gallon of fermentation broth yields about 0.4 lb to about 0.6 lb freeze dried powder. In another instance, one gallon of fermentation broth yields about 0.5 lb freeze dried powder.

In a further embodiment, the resulting dry products may be further processed, such as by milling or granulation, with or without the addition of inerts or additives to achieve specific particle sizes or physical formats or physical properties desirable for agricultural applications.

In addition to the use of whole broth or broth concentrate, cell-free preparations of fermentation broth of the novel variants and strains of Streptomyces of the present invention can be obtained by any means known in the art, such as extraction, centrifugation and/or filtration of fermentation broth. Those of skill in the art will appreciate that so-called cell-free preparations may not be devoid of cells but rather are largely cell-free or essentially cell-free, depending on the technique used (e.g., speed of centrifugation) to remove the cells. The resulting cell-free preparation may be dried and/or formulated with components that aid in its application. Concentration methods and drying techniques described above for fermentation broth are also applicable to cell-free preparations.

In certain aspects, the fermentation product further comprises a formulation ingredient. The formulation ingredient may be a wetting agent, extender, solvent, spontaneity promoter, emulsifier, dispersant, frost protectant, thickener, and/or an adjuvant. In one embodiment, the formulation ingredient is a wetting agent. In other aspects, the fermentation product is a freeze-dried powder or a spray-dried powder.

Compositions of the present invention may include formulation ingredients added to compositions of the present invention to improve recovery, efficacy, or physical properties and/or to aid in processing, packaging and administration. Such formulation ingredients may be added individually or in combination.

The formulation ingredients may be added to compositions comprising cells, cell-free preparations and/or metabolites to improve efficacy, stability, and physical properties, usability and/or to facilitate processing, packaging and end-use application. Such formulation ingredients may include carriers, inerts, stabilization agents, preservatives, nutrients, or physical property modifying agents, which may be added individually or in combination. In some embodiments, the carriers may include liquid materials such as water, oil, and other organic or inorganic solvents and solid materials such as minerals, polymers, or polymer complexes derived biologically or by chemical synthesis. In some embodiments, the formulation ingredient is a binder, adjuvant, or adhesive that facilitates adherence of the composition to a plant part, such as leaves, seeds, or roots. See, for example, Taylor, A. G., et al., “Concepts and Technologies of Selected Seed Treatments” Annu. Rev. Phytopathol. 28: 321-339 (1990). The stabilization agents may include anti-caking agents, anti-oxidation agents, anti-settling agents, antifoaming agents, desiccants, protectants or preservatives. The nutrients may include carbon, nitrogen, and phosphorus sources such as sugars, polysaccharides, oil, proteins, amino acids, fatty acids and phosphates. The physical property modifiers may include bulking agents, wetting agents, thickeners, pH modifiers, rheology modifiers, dispersants, disintegrants, adjuvants, surfactants, film-formers, hydrotropes, builders, antifreeze agents or colorants. In some embodiments, the composition comprising cells, cell-free preparation and/or metabolites produced by fermentation can be used directly with or without water as the diluent without any other formulation preparation. In a particular embodiment, a wetting agent, or a dispersant, is added to a fermentation solid, such as a freeze-dried or spray-dried powder. A wetting agent increases the spreading and penetrating properties, or a dispersant increases the dispersibility and solubility of the active ingredient (once diluted) when it is applied to surfaces. Exemplary wetting agents are known to those of skill in the art and include sulfosuccinates and derivatives, such as MULTIWET™ MO-70R (Croda Inc., Edison, N.J.); siloxanes such as BREAK-THRU® (Evonik, Germany); nonionic compounds, such as ATLOX™ 4894 (Croda Inc., Edison, N.J.); alkyl polyglucosides, such as TERWET® 3001 (Huntsman International LLC, The Woodlands, Tex.); C12-C14 alcohol ethoxylate, such as TERGITOL® 15-S-15 (The Dow Chemical Company, Midland, Mich.); phosphate esters, such as RHODAFAC® BG-510 (Rhodia, Inc.); and alkyl ether carboxylates, such as EMULSOGEN™ LS (Clariant Corporation, North Carolina).

In some embodiments, the formulation inerts are added after concentrating fermentation broth and during and/or after drying.

The compositions according to the present invention can be used as such or, depending on their particular physical and/or chemical properties, in the form of their formulations or the use forms prepared therefrom, such as aerosols, capsule suspensions, cold-fogging concentrates, warm-fogging concentrates, encapsulated granules, fine granules, flowable concentrates for the treatment of seed, ready-to-use solutions, dustable powders, emulsifiable concentrates, oil-in-water emulsions, water-in-oil emulsions, macrogranules, microgranules, oil-dispersible powders, oil-miscible flowable concentrates, oil-miscible liquids, foams, pastes, pesticide coated seed, suspension concentrates, suspoemulsion concentrates, soluble concentrates, suspensions, wettable powders, soluble powders, dusts and granules, water-soluble and water-dispersible granules or tablets, water-soluble and water-dispersible powders for the treatment of seed, wettable powders, natural products and synthetic substances impregnated with active ingredient, and also microencapsulations in polymeric substances and in coating materials for seed, and also ULV cold-fogging and warm-fogging formulations.

In some embodiments, the composition is formulated as a water-dispersible granule or a wettable powder. In solid formulations, the composition of the present invention may contains at least about 1×10⁶ colony forming units (CFU), at least about 1×10⁷ CFU, at least about 1×10⁸ CFU, at least about 1×10⁹ CFU, at least about 1×10¹⁰ CFU, at least about 1×10¹¹ CFU, or at least about 1×10¹² CFU of the microorganism (e.g., Streptomyces microflavus NRRL B-50550 or a phytophagous-miticidal mutant strain thereof)/gram.

The present invention encompasses fermentation broths containing gougerotin at a concentration of at least about 1 g/L. In some embodiments such whole broth cultures come from gougerotin-producing strains of Streptomyces. In a particular embodiment, such gougerotin-producing strain is Streptomyces microflavus, Streptomyces puniceus, or Streptomyces graminearus. In another embodiment, the gougerotin-producing strain is S. griseus, S. anulatus, S. fimicarius, S. parvus, S. lavendulae, S. alboviridis, or S. puniceus. In yet another particular embodiment, such gougerotin-producing strain is Streptomyces graminearus CGMCC 4.506, deposited at China General Microbiological Culture Collection Center CGMCC.

Fermentation broths containing at least about 1 g/L gougerotin may be obtained in several ways, such as fermentation optimization and/or mutagenesis of a parent gougerotin-producing strain in order to attain a mutant strain that produces higher levels of gougerotin than the parent strain.

Thus, the present invention also encompasses a method of producing a fermentation broth of a gougerotin producing Streptomyces strain, wherein the fermentation broth contains at least about 0.5 g/L gougerotin. The method comprises cultivating the Streptomyces strain in a culture medium that contains a digestible carbon source and a digestible nitrogen source under aerobic conditions, wherein the culture medium contains a precursor to gougerotin, such as cytosine; a nucleobase; and/or an amino acid at a concentration effective to achieve a gougerotin concentration of at least 0.5 g/L.

In some embodiments, the Streptomyces strain is cultivated in the culture medium until the culture medium contains gougerotin in a concentration of at least about 0.5 g/L, of at least about 1 g/L, of at least about 2 g/L, of at least about 3 g/L, of at least about 4 g/L, of at least about 5 g/L, of at least about 6 g/L, of about at least 7 g/L or of at least about 8 g/L gougerotin.

In other embodiments, the Streptomyces strain is cultivated in the culture medium until the culture medium contains gougerotin in a concentration ranging from about 0.5 g/L to about 25 g/L gougerotin, meaning the fermentation broth contains gougerotin in a concentration ranging typically ranging from about 0.5 g/L to about 15 g/L gougerotin after completion of the fermentation of rom about 0.5 mg/g to about 15 mg/g gougerotin.

In this context it is noted that the amino acid that is added at a concentration effective to achieve a gougerotin concentration of at least about 0.5 g/L or at least about 1 g/L is provided to the culture medium as a separate individual component in a defined concentration and not part of a composition such as a yeast extract or a protein hydrolysate (for example, casein hydrolysate, soy flour hydrolysate, soy peptone, soy acid hydrolysate, to name only a few) in which amino acids may be present in a mixture with other compounds such as oligopeptides and partially hydrolyzed proteins. Thus, by “a concentration effective to achieve a gougerotin concentration of at least 1 g/L” in the fermentation broth is meant a concentration of an amino acid in the culture medium that is specifically chosen to provide such a gougerotin concentration. In some embodiments, the concentration effective to achieve the desired gougerotin concentration is a concentration of the amino acid in the culture medium of at least about 1 g/L. This “effective concentration” may thus be higher than 2 g/L and may, for example, range from about 2 g/L to about 15 g/L. The concentration may be about 3 g/L, about 4 g/L, about 5 g/L, about 6 g/L, about 7 g/L, about 8 g/L, about 9 g/L, about 10 g/L, about 11 g/L, about 12 g/L, about 13 g/L, or about 14 g/L.

The amino acid may be any amino acid which provides for a concentration of gougerotin of at least about 0.5 g/L or a higher concentration such as at least about 1 g/L, at least about 2 g/L, at least about 3 g/L, at least about 4 g/L, at least about 5 g/L, at least about 6 g/L, method of any of claims 6 to 8. In some embodiments the amino acid is glycine, L-glutamic acid, L-glutamine, L-aspartic acid, L-serine, or a mixture thereof. In some embodiments the culture medium contains glycine at a concentration of about 5 g/L to about 15 g/L, whereas in other embodiments the culture medium contains glutamic acid in an initial concentration of about 5 g/L to about 15 g/L. It is also possible that the culture medium contains both glycine and L-glutamic acid (or L-glutamine) in a concentration of about 5 g/L to about 15 g/L.

Any carbon source that is digestible (and thus available) for Streptomyces strains can be used in the method of producing a fermentation broth (or fermentation method) as described here. Examples of suitable carbon sources include glucose, fructose, mannose, galactose, sucrose, maltose, lactose, molasses, starch (as an example for a polysaccharide), dextrin, maltodextrin (as an example of an oligosaccharide) or glycerin, to name only a few. The total initial concentration of the carbon source (or sources) may be any concentration that provides a suitable growth of Streptomyces and production of the desired concentration of gougerotin and may be determined experimentally (determining the final concentration of gougerotin in the fermentation broth dependent from the concentration of the used carbon source(s)). The total initial carbon source concentration may, for example, be in the range of about 10 g/L to about 150 g/L, for example, about 10 g/L, about 20 g/L, about 30 g/L, about 40 g/L, about 50 g/L, about 60 g/L, about 70 g/L, about 80 g/L, about 90 g/L, about 100 g/L, about 110 g/L or about 120 g/L. In some embodiments, the carbon source might be a mixture of two or more carbon sources, for example, a mixture of glucose with a polysaccharide such as starch, a mixture of glucose and an oligosaccharide such as dextrin or maltodextrin or a mixture of glucose, starch and dextrin. In some embodiments the culture medium contains as carbon source a mixture of glucose and an oligosaccharide. The oligosaccharide may be maltodextrin or dextrin. In such embodiments, the initial maltodextrin concentration in the culture medium may be about 50 g/L to about 100 g/L or about 60 g/L to about 80 g/L. The initial glucose concentration in the culture medium may be about 20 g/L to about 80 g/L, for example, about 30 g/L, about 40 g/L, about 50 g/L, about 60 g/L or about 70 g/L. In other embodiments in which glucose is used as carbon source with maltodextrin or dextrin, the glucose concentration may be about 20 g/L to 60 g/L or about 30 g/L to about 50 g/L.

Any nitrogen source that is digestible can be used in the fermentation process described here. The nitrogen source can be a single source or a mixture of sources. In illustrative embodiments the nitrogen source is (at least partially) selected from the group consisting of soy peptone, soy acid hydrolysate, soy flour hydrolysate, casein hydrolysate, yeast extract, and mixtures thereof. The total initial concentration of the nitrogen source(s) may be any concentration that provides a suitable growth of Streptomyces and production of the desired concentration of gougerotin and may be determined experimentally. Suitable total concentrations in the culture medium may, for example, be in the range of about 10 g/L to about 60 g/L, for example, about 20 g/L, about 30 g/L, about 40 g/L, about 50 g/L. In illustrative embodiments, the nitrogen source may be a mixture of casein hydrolysate and soy flour hydrate or a mixture of yeast extract and soy acid hydrolysate, wherein for example the yeast extract is used in the culture medium in a concentration (or amount) of 10 g/L and the soy acid hydrolysate is used in a concentration/amount of 20 g/L.

The culture medium can further contain a calcium source such as calcium chloride, or calcium carbonate. If present, the culture medium may contain a calcium source such as calcium carbonate in an initial concentration of about 1 g/L to 3 g/L.

In this context, it is noted that concentrations of all ingredients of the culture medium are given as concentration at the beginning of the fermentation (initial concentrations) unless indicated otherwise. The concentrations are based on the post inoculation volume that is used for the fermentation. The initial concentrations as given here can either be maintained during the fermentation by continuous nutrient feeding or, alternatively, the ingredients (carbon source, nitrogen source, amino acid) can be added only at the beginning of the fermentation. However, the pH of the culture medium/fermentation broth is typically continuously monitored and controlled by addition of a suitable acid (such as sulfuric acid or citric acid) and/or of a suitable base (such as sodium hydroxide or ammonia solution or potassium hydroxide). An appropriate pH can be determined empirically. In typical embodiments the pH of the culture medium/fermentation broth is in range of 6.5 to 7.5, for example, 6.8 to 7.0. Also process parameters such as temperature and aeration rate are usually controlled over the course of fermentation process. Since the cultivation of the Streptomyces strain is carried out under aerobic conditions, the fermentation broth is typically aerated with air, oxygen enriched air or if necessary, pure oxygen. The temperature is usually chosen to be within a range of 20° C. to 30° C., however higher temperatures are also contemplated herein. Standard fermentation reagents such as antifoam agents may also be added continuously. The production of the fermentation broth can be carried out using conventional large-scale microbial fermentation processes, such as submerged fermentation, solid state fermentation or liquid surface culture, including the methods described, for example, in U.S. Pat. No. 3,849,398; British Patent No. GB 1 507 193; Toshiko Kanzaki et al., Journal of Antibiotics, Ser. A, Vol. 15, No. 2, June 1961, pages 93 to 97; or Toru Ikeuchi et al., Journal of Antibiotics, (September 1972), pages 548 to 550.

Any gougerotin producing Streptomcyes strain can be used for producing the gougerotin containing fermentation broth disclosed herein. In illustrative embodiments the Streptomcyes strain is a Streptomyces microflavus strain, Streptomcyes puniceus strain or a Streptomyces graminearus strain. The Streptomyces microflavus strain may, for example, be Streptomyces microflavus strain NRRL B-50550 or a phytophagous-miticidal and/or fungicidal mutant strain derived therefrom. In addition, parent bacterial strains, such as various Streptomycetes (including, but not limited to, Streptomyces microflavus, Streptomyces puniceus and Streptomyces graminearus) and Bacilli, capable of producing gougerotin, even at low levels, may be mutagenized for enhanced gougerotin production. Example 13 describes one way to produce such mutants and resulting fermentation broths containing at least 1 g/L gougerotin.

Selection of specific carbon and nitrogen sources and other nutrients during fermentation may be used to optimize the production of gougerotin. Suitable carbon sources for enhancing gougerotin production are starch, maltodextrin, dextrin, sugars and glucose. In a specific embodiment a combination of glucose and an oligosaccharide is used as the carbon source and/or procures. Suitable nitrogen sources for enhancing gougerotin production are soy protein hydrolysate, casein hydrolysate, soy peptone, yeast extract, and other nitrogen sources that are less nutrient rich. Other suitable nitrogen sources include amino acids and/or precursors to gougerotin such as glycine, glutamic acid, including L-glutamic acid, aspartic acid, including L-aspartic acid, serine, including L-serine, and cytosine. Cytosine may be added as part of a media component that has a high concentration of cytosine, such as a yeast extract having high nucleobase content. Examples of fermentation media capable of producing a fermentation broth having an increased level of gougerotin are provided in the Examples.

In another embodiment, the fermentation products (e.g., fermentation broth or fermentation solid) of the present invention have potency of at least 40%, at least 50%, or at least 60%, wherein the potency is measured as follows. Dilute the fermentation product in a water surfactant solution (using the amount of surfactant recommended on the surfactant product label) to obtain a solution that is 5% whole broth (or whole broth equivalent, as described below, if dealing with a fermentation solid derived from whole broth). Apply the diluted solution to the top and bottom surfaces of a leaf (such as the leaf of a lima bean) until both surfaces are wet, but do not apply to run-off. Allow plants to dry and then infest with 10-20 two-spotted spider mites (Tetranychus urticae Koch). Four days after treatment, inspect the treated leaves and count live and dead adult females and deutonmphs on the leaves. Use the Sun-Shepard formula to calculate potency (i.e., corrected mortality). Corrected %=100 (% reduction in the treated plot±% change in untreated population)/(100±% change in untreated population). In this application, potency calculated by the above-described method will be referred to as “Spider Mite Potency.”

In some embodiments the compositions of the present invention are used to treat a wide variety of agricultural and/or horticultural crops, including those grown for seed, produce, landscaping and those grown for seed production. Representative plants that can be treated using the compositions of the present invention include but are not limited to the following: brassica, bulb vegetables, cereal grains, citrus, cotton, cucurbits, fruiting vegetables, leafy vegetables, legumes, oil seed crops, peanut, pome fruit, root vegetables, tuber vegetables, corm vegetables, stone fruit, tobacco, strawberry and other berries, and various ornamentals.

The compositions of the present invention may be administered as a foliar spray, as a soil treatment, and/or as a seed treatment/dressing. When used as a foliar treatment, in one embodiment, about 1/16 to about 5 gallons of whole broth are applied per acre. When used as a soil treatment, in one embodiment, about 1 to about 15 gallons or about 1 to about 5 gallons of whole broth are applied per acre or about 0.1 mg to about 14 mg, or about 0.2 mg to about 10 mg, or about 0.2 mg to about 8 mg fermentation product, such as a freeze dried product, depending on the size of the seeds to be treated and the concentration of colony forming units in the fermentation product. When used for seed treatment about 1/32 to about ¼ gallons of whole broth are applied per acre. For seed treatment, the end-use formulation contains at least 1×10⁸ colony forming units per gram.

In some embodiments, application of the compositions of the present invention to plants, plant parts or plant loci is preceded by identification of a locus in need of treatment.

A fermentation product, such as a whole broth culture or a fermentation solid, including a freeze-dried powder, of the microorganism (e.g., Streptomyces microflavus NRRL B-50550 or a phytophagous-miticidal and/or fungicidal mutant strain thereof)/mL is diluted and applied to plants foliarly. Application rates are provided in gallons or pounds per acre and can be adjusted proportionally to smaller applications (such as the microplot trials described in the Examples). As described in the Examples, for larger applications, the fermentation product is diluted in 100 gallons of water before application. In one embodiment, about 0.5 gallons to about 15 gallons, about 1 gallon to about 12 gallons, about 2.5 gallons to about 12.5 gallons, about 5 gallons to about 10 gallons, or about 1.25 gallons to about 10 gallons whole broth culture (diluted in water and, optionally, a surfactant) are applied to plants foliarly per acre. In another embodiment, about 0.05 lbs to about 10 pounds of freeze-dried powder, about 0.05 lbs to about 5 lbs, about 0.1 lbs to about 10 lbs, about 0.25 lbs to about 7.5 lbs., about 0.4 lbs to about 7 pounds, or about 0.4 lbs to about 6 lbs (diluted in water and, optionally, a surfactant) are applied to plants foliarly per acre. In a particular instance, the fermentation product has Spider Mite Potency of at least about 40%, at least about 50% or at least about 60%. In another instance, the fermentation product is a fermentation powder (including spray-dried or freeze-dried powder) having about 0.25% to about 20% gougerotin, about 0.5% to about 15% gougerotin, about 1% to about 12% gougerotin, or about 2% to about 10% gougerotin, where all percentages are weight by weight. In another instance, the fermentation product is a fermentation broth having about 0.01% to about 0.5% gougerotin, weight by weight.

In a particular embodiment, about 0.5 pounds of fermentation product, such as freeze-dried powder or spray-dried powder, (diluted in water and, optionally, a surfactant) are applied to plants foliarly per acre. In these embodiments, the end-use formulation is based on a starting fermentation broth containing at least about 1×10⁶ colony forming units per mL, at least about 1×10⁷ colony forming units per mL, at least about 1×10⁸ colony forming units per mL, at least about 1×10⁹ colony forming units per mL, at least about 1×10¹⁰ colony forming units per mL, or at least about 1×10¹¹ colony forming units per mL. In another example, this fermentation product contains at least about 1% by weight gougerotin, at least about 2% by weight gougerotin, at least about 3% by weight gougerotin, at least about 4% by weight gougerotin, at least about 5% by weight gougerotin, at least about 6% by weight gougerotin, at least about 7% by weight gougerotin, at least about 8% by weight gougerotin, at least about 9% by weight gougerotin, at least about 10% by weight gougerotin, at least about 15% by weight gougerotin, or at least about 20% by weight gougerotin.

### Deposit Information

A sample of a Streptomyces microflavus strain of the invention has been deposited with the Agricultural Research Service Culture Collection located at the National Center for Agricultural Utilization Research, Agricultural Research Service, U.S. Department of Agriculture, 1815 North University Street, Peoria, Ill. 61604, U.S.A., under the Budapest Treaty on Aug. 19, 2011 and has been assigned the following depository designation: NRRL B-50550.

A sample of a mutant of Streptomyces microflavus strain NRRL B-50550 (designated herein as Streptomyces microflavus strain M and also known as AQ6121.002 or AQ32392) has been deposited with the Agricultural Research Service Culture Collection located at the National Center for Agricultural Utilization Research, Agricultural Research Service, U.S. Department of Agriculture, 1815 North University Street, Peoria, Ill. 61604, U.S.A., under the Budapest Treaty on Sep. 27, 2013 and has been assigned the following depository designation: NRRL B-50875. This strain has also been deposited with the American Type Culture Collection located at 10801 University Boulevard Manassas, Va. 20110, U.S.A., under the Budapest Treaty on Oct. 8, 2013, and has been assigned the following patent deposit designation: PTA-120616. This strain has also been deposited with the International Depositary Authority of Canada located at 1015 Arlington Street Winnipeg, Manitoba Canada R3E 3R2, on Oct. 9, 2013 and has been assigned Accession No. 091013-02.

A sample of a Streptomyces puniceus strain referred to herein as Streptomyces puniceus strain A (and also known as AQ7439) has been deposited with the American Type Culture Collection located at 10801 University Boulevard Manassas, Va. 20110, U.S.A., under the Budapest Treaty on Oct. 8, 2013, and has been assigned the following patent deposit designation: PTA-120615. This strain has also been deposited with the International Depositary Authority of Canada located at 1015 Arlington Street Winnipeg, Manitoba Canada R3E 3R2, on Oct. 9, 2013, and has been assigned Accession No. 091013-01.

Samples of five mutant strains identified during a screen of genome shuffled derivative strains of Streptomyces microflavus strain NRRL B-50550 (see Example 17) were deposited with the Agricultural Research Service Culture Collection located at the National Center for Agricultural Utilization Research, Agricultural Research Service, U.S. Department of Agriculture, 1815 North University Street, Peoria, Ill. 61604, U.S.A., under the Budapest Treaty on Apr. 1, 2014, and have been assigned the following accession numbers: NRRL B-50954, NRRL B-50955, NRRL B-50956, NRRL B-50957, and NRRL B-50958.

All publications, patents and patent applications, including any drawings and appendices therein, are incorporated by reference to the same extent as if each individual publication, patent or patent application was specifically and individually indicated to be incorporated by reference.

The following examples are given for purely illustrative and non-limiting purposes of the present invention.

### EXAMPLES

### Example 1

### Selection of Streptomyces microflavus NRRL B-50550

Strains were taken from an internal collection of strains and initial screening tests were conducted to determine efficacy of potential candidates strain against two-spotted spider mites (“TSSM”), which are a model organism commonly used to screen for general miticidal activity. Microorganisms were selected initially for properties that favor laboratory or artificial cultivation, such as variants that grow rapidly on an agar plate. Culture stocks of the selected strains were grown in suitable media for the respective strain, such as the Medium 1 and Medium 2 described in Example 2. The resulting fermentation products (whole broths) were diluted to a 25% solution using water and 0.03% of the surfactant BREAK-THRU FIRST CHOICE® polyether-polymethylsiloxane-copolymer. Thereafter, 8 mL of the diluted fermentation products were applied to run-off to the top and bottom of lima bean leaves of two plants (the lima bean plants were 1 to 1.5 weeks old). After such treatment, plants were infested on the same day with 50-100 TSSM and left in the greenhouse for five days. On the fifth day plants were assessed for presence of mites and eggs on a scale of 1 to 4. The miticide AVID® (abamectin, Syngenta) was used as positive control. For mites and eggs, 1 indicates 100% mortality, 1.5 indicates 90% to 95% mortality, 2.0 represents 75% to 90% mortality; 2.5 represents 40% to 55% mortality; 3.0 represents 20% to 35% mortality and 4.0 represents 0% to 10% mortality. Besides NRRL B-50550, other Streptomcyes strains and some Bacillus strains were found to be active against mites.

For further selection, amongst other activities, the UV stability and translaminar activity of the screened strains was examined since an acaracide should be stable to UV light and possess translaminar activity in order to be effective in field applications.

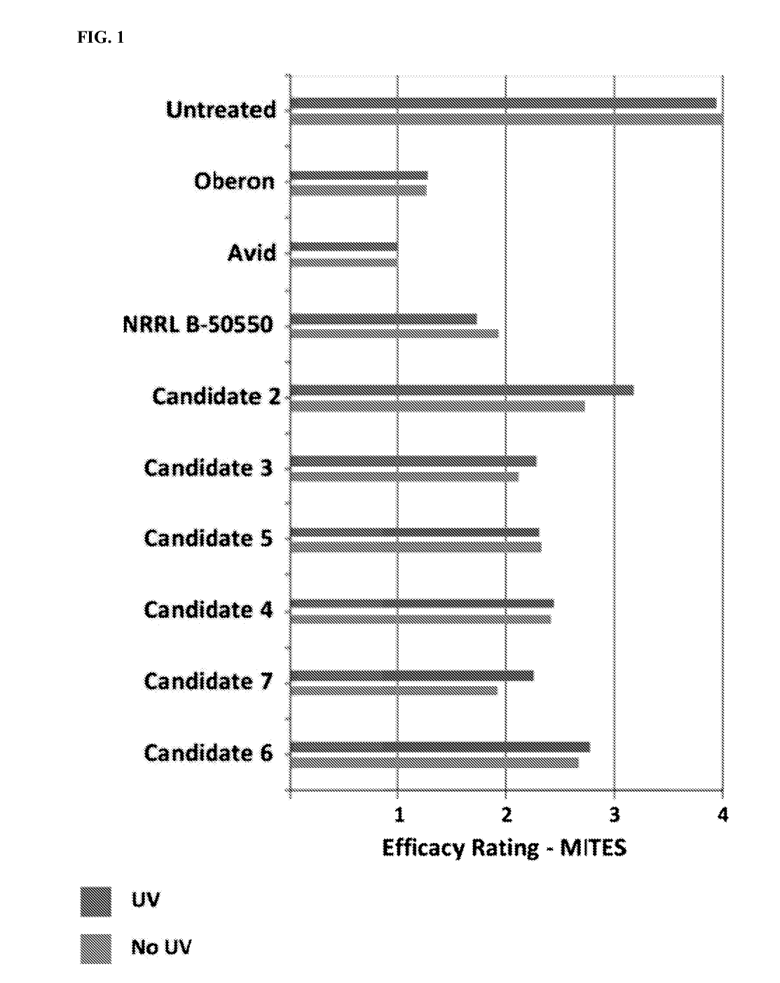

For assessment of the UV stability the above-described 25% dilutions of the fermentation products were sprayed on the upper surface of lima bean plants. After such treatment, plants were infested on the same day with 50-100 TSSM, exposed to UV light for 24 hrs and left in the greenhouse for five days. The mites were confined to the adaxial (upper) surface of the leaves by means of a Vaseline ring which was applied to the leaf and served as an impassable boundary to the mites. On the fifth day plants were assessed for presence of mites and eggs on a scale of 1 to 4, as described above. The miticides AVID® (abamectin, Syngenta) and OBERON® (spiromesifen, Bayer CropScience AG) were used as controls. Results are shown in FIG. 1. The fermentation product of the strain NRRL B-50550 showed the best UV stability of all strains tested.

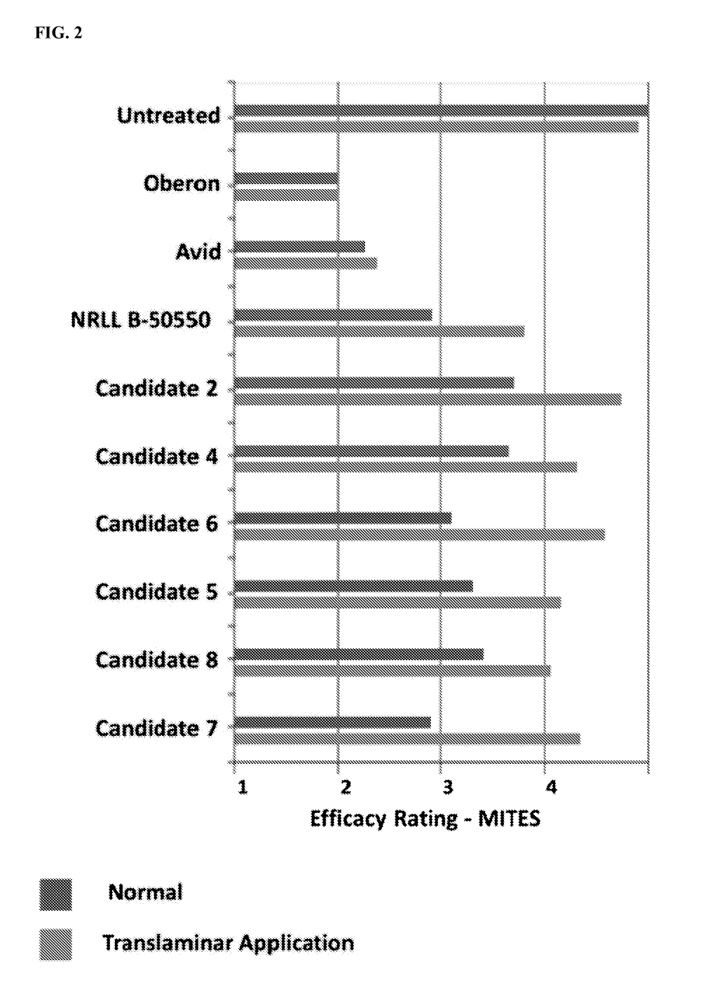

For assessment of the translaminar activity the strains were cultured as described above and the resulting whole broth was diluted using water and 0.35% surfactant and applied to run-off to the lower surface of lima bean leaves on two plants. The upper surface of the treated leaves was infested one day after treatment with 50-100 TSSM, which were placed on the upper surface of the leaves and contained using a Vaseline ring/physical barrier as described above. On the sixth day plants were assessed for presence of mites and eggs on the above-described scale of 1 to 4. The miticides AVID® (abamectin, Syngenta) and OBERON® (spiromesifen, Bayer CropScience AG) were used as controls. Results are shown in FIG. 2. The fermentation product of the strain NRRL B-50550 showed the best translaminar activity of all strains tested.

### Example 2

### Activity Against Spider Mites

Further tests were conducted to more closely determine the efficacy of Streptomyces microflavus NRRL B-50550 against two-spotted spider mites (“TSSM”). Culture stocks of Streptomyces microflavus NRRL B-50550 were grown in 1 L shake flasks in Medium 1 or Medium 2 at 28° C. for 5 days. Medium 1 was composed of 2.0% starch, 1.0% dextrose, 0.5% yeast extract, 0.5% casein hydrolysate and 0.1% CaCO₃. Medium 2 was composed of 2% ProFlo cotton seed meal, 2% malt extract, 0.6% KH₂PO₄ and 0.48% K₂HPO₄. The resulting fermentation products were diluted to a 25% solution using water and 0.03% surfactant BREAK-THRU FIRST CHOICE® (polyether-polymethylsiloxane-copolymer), and 6 mL were applied to run-off to the top and bottom of lima bean leaves of two plants. After such treatment, plants were infested on the same day with 50-100 TSSM and left in the greenhouse for five days. On the sixth day plants were assessed for presence of mites and eggs on a scale of 1 to 4. The miticide AVID® (abamectin, Syngenta) was used as positive control. For mites and eggs, 1 indicates 100% mortality, 1.5 indicates 90% to 95% mortality, 2.0 represents 75% to 90% mortality; 2.5 represents 40% to 55% mortality; 3.0 represents 20% to 35% mortality and 4.0 represents 0% to 10% mortality. Results are shown in Table 1 below. Both fermentation products of Streptomyces microflavus NRRL B-50550 resulted in a mortality of mites of 90% or greater.

TABLE 1

| Fermentation Product | Mites | Eggs |
|---|---|---|
| NRRL B-50550 Medium 1 | 1.25 | 1.00 |
| NRRL B-50550 Medium 2 | 1.50 | 1.50 |
| Positive Control (AVID ® | 1.00 | 1.00 |
| abamectin, EC - 5.7 ppm) | | |
| Untreated Control | 3.75 | 4.00 |

Field trials against Pacific spider mite in almond, Pacific spider mite in grapes, and two-spotted spider mite in strawberry, confirmed the above greenhouse results. Results of field trials against Pacific spider mite in almonds are reported in Tables 2-4, below. The miticide AGRI-MEK® (abamectin, Syngenta) was used as positive control. Shake flasks containing Medium 1 were inoculated with frozen cultures of NRRL B-50550 and grown 1-2 days at 28-30° C. The resulting fermentation product was used to seed a 20 L bioreactor containing the following media: 6.0% starch, 3.0% dextrose, 1.5% yeast extract and 1.5% casein hydrolysate and 0.3% calcium carbonate. This medium was fermented at between 28° C. for 7 days. The resulting whole broth was used to create a freeze dried powder (“FDP”) that was mixed with an adjuvant, BREAK-THRU FIRST CHOICE® (polyether-polymethylsiloxane-copolymer), at 0.03% and then used in the trial.

TABLE 2
Activity Against Adult Mites

| No. Adult Mites/Leaf | 0 DAT | 3 DAT | 7 DAT | 14 DAT |
|---|---|---|---|---|
| Untreated | 9.3 | 8.8 | 10.5 | 5.8 |
| NRRL B-50550 FDP 0.63 lb/acre | 15.0 | 0.8 | 0.0 | 0.0 |
| NRRL B-50550 FDP1.25 lb/acre | 13.5 | 1.3 | 0.8 | 0.3 |
| NRRL B-50550 FDP 2.5 lb/acre | 15.0 | 0.8 | 0.0 | 0.0 |
| NRRL B-50550 FDP 5 lb/acre | 16.8 | 0.0 | 0.3 | 0.0 |
| Standard (AGRI-MEK ® | 7.0 | 0.0 | 0.0 | 0.0 |
| abamectin 0.15 EC at 16 fl. | | | | |
| oz/acre) | | | | |

TABLE 3
Activity Against Juvenile Mites

| No. Juvenile Mites/Leaf | 0 DAT | 3 DAT | 7 DAT | 14 DAT |
|---|---|---|---|---|
| Untreated | 23.8 | 24.3 | 29.8 | 12.5 |
| NRRL B-50550 FDP 0.63 lb/acre | 43.0 | 3.0 | 1.8 | 0.0 |
| NRRL B-50550 FDP 1.25 lb/acre | 31.5 | 2.0 | 1.3 | 0.0 |
| NRRL B-50550 FDP 2.5 lb/acre | 41.8 | 2.0 | 0.8 | 0.0 |
| NRRL B-50550 FDP 5 lb/acre | 39.3 | 0.8 | 0.3 | 0.0 |
| Standard (AGRI-MEK ® | 37.5 | 0.5 | 1.0 | 0.3 |
| abamectin 0.15 EC at 16 fl. | | | | |
| oz/acre) | | | | |

TABLE 4
Activity against Mite Eggs

| No. Mite Eggs/Leaf | 0 DAT | 3 DAT | 7 DAT | 14 DAT |
|---|---|---|---|---|
| Untreated | 26.8 | 21.0 | 19.8 | 9.5 |
| NRRL B-50550 FDP 0.63 lb/acre | 23.5 | 4.8 | 5.5 | 0.0 |
| NRRL B-50550 FDP 1.25 lb/acre | 16.3 | 3.0 | 2.3 | 0.5 |
| NRRL B-50550 FDP 2.5 lb/acre | 29.0 | 3.3 | 2.8 | 0.0 |
| NRRL B-50550 FDP 5 lb/acre | 33.8 | 5.0 | 3.3 | 0.8 |
| Standard (AGRI-MEK ® | 22.3 | 5.8 | 1.3 | 0.3 |
| abamectin 0.15 EC at 16 fl. | | | | |
| oz/acre) | | | | |

### Example 3

### Field Activity Against Citrus Mite

Field trials were conducted to determine efficacy of NRRL B-50550 against citrus rust mites (Phyllocoptruta oleivora) on Valencia oranges. Shake flasks containing Medium 1 (see Example 2) were inoculated with frozen cultures of NRRL B-50550 and grown 1-2 days at 20-30° C. This was repeated. The resulting fermentation product was used to seed a 20 L bioreactor containing the following media: 6.0% starch, 3.0% dextrose, 1.5% yeast extract, 2.0% soy acid hydrolysate, 0.6% glycine, and 0.2% calcium carbonate. This medium was fermented at between 28° C. for 8 days. The resulting whole broth was used to create a freeze dried powder (“FDP”) used in the following trials. The freeze dried powder was diluted in water and applied at 100 gal/acre at the rates shown in Table 5 below. The miticide ENVIDOR® (spirodiclofen, Bayer CropScience, Germany) was used as positive control. In treatments 1-3, the BREAK-THRU FIRST CHOICE® adjuvant (polyether-polymethylsiloxane-copolymer, see above) was added at 0.66% v/v. The fermentation product applied at a rate of 0.625 lb/A showed a better miticidal activity than ENVIDOR® spirodiclofen applied at a rate of 16-fl oz/A.

TABLE 5

| | Treatment | Rate | | No. Mites/cm² Fruit |
|---|---|---|---|---|
| 1. | NRRL B-50550 FDP | 0.625 | lb/A | 0.29 |
| 2. | NRRL B-50550 FDP | 1.25 | lb/A | 1.43 |
| 3. | NRRL B-50550 FDP | 2.5 | lb/A | 0.78 |
| 4. | NRRL B-50550 + | 2.5 lb/A + | | 0.76 |
| | 435 Oil | 5 gal/A | | |
| 5. | ENVIDOR ® 2SC | 16 | fl oz/A | 0.41 |
| | (spirodiclofen) | | | |

| | | | |
|---|---|---|---|
| 6. | Untreated Check | — | 13.09 |

### Example 4

### Activity Against Other Mites

Studies have shown that NRRL B-50550 is active against various other mites including eriophyid (russet) mites and broad mites. Fermentation broth was prepared as it was for the field trials described in Example 2. The resulting fermentation broth was diluted to various concentrations using water and 0.35% surfactant and 10 mL of the diluted broth applied to run-off to the top and bottom of lima bean leaves on two plants. Plants were infested on the day of treatment and assessed for presence of russet mites on the scale described above 6 days after treatment. A score of four indicated no control and presence of at least 100 russet mites at time of assessment. The miticide AVID® (abamectin) was used as positive control. The results are presented in Table 6.

TABLE 6

| | Rating - New | Rating - Old |
|---|---|---|
| Treatment | Leaves | Leaves |
| NRRL B-50550 WB 12.50% | 1.58 | 1.50 |
| NRRL B-50550 WB 6.25% | 1.75 | 1.92 |
| NRRL B-50550 WB 3.12% | 2.42 | 2.67 |
| NRRL B-50550 WB 1.56% | 2.75 | 3.17 |
| Untreated | 4.00 | 4.00 |
| AVID ® (EC) 1% (abamectin) | 1.00 | 1.00 |

### Example 5

### Residual Activity

Other studies revealed that NRRL B-50550 has residual activity. Shake flasks containing Medium 1 of Example 2 were inoculated with Luria broth based cultures of NRRL B-50550 (which had been inoculated with a frozen culture of NRRL B-50550) and grown 1-2 days at 28° C. The resulting fermentation product was used to seed a 20 L bioreactor containing the following media: 8.0% dextrose, 1.5% yeast extract, 1.5% casein hydrolysate and 0.1% calcium carbonate. This medium was fermented at between 28° C. for 7-8 days. The resulting fermentation product was diluted to 3.13% solution using water and 0.35% surfactant, and 8 mL of the diluted broth were applied to run-off to the top and bottom of lima bean leaves on two plants. Plants were infested six days after such treatment with 50-100 TSSM and assessed for presence of mites and eggs on the scale described above 12 days after treatment. The miticide AVID® (abamectin) was used as positive control. Results are shown in Table 7 below.

TABLE 7

| Fermentation Product | Mites | Eggs |
|---|---|---|
| NRRL B-50550 WB 3.13% | 1.12 | 1.31 |
| Positive Control (AVID ® - abamectin | 1.00 | 1.00 |
| 0.4 μL/10 mL) | | |
| Untreated Control | 4.00 | 4.00 |

Beyond the effects on mites initially exposed to treated plants, the effects on mites that might migrate onto treated leaves at later time points was also evaluated. All plants were treated on day zero with either 6.25% or 1.56% whole broth produced in a manner similar to that described in Example 13. Then, mites were added to groups of plants at one-week intervals after treatment. This set of treatments included other miticides for comparison. Mites were added for each of five weeks after treatment. Activity was maintained over the five week period and the rate of activity decrease was similar to the OBERON® (spiromesifen) product and slightly greater than the AVID® product. This study also showed that when the primary leaves of lima bean plants were treated, leaves that emerged later were not protected.

### Example 6

### Translaminar Activity

Studies were conducted to determine whether NRRL B-50550 has translaminar activity. Whole broth was prepared as described in Example 5. The resulting whole broth was diluted using water and 0.35% surfactant, and 10 mL of the diluted broth were applied to run-off to the lower surface of lima bean leaves on two plants. The upper surface of the treated leaves was infested one day after treatment with 50-100 TSSM, which were placed on the upper surface of the leaves and contained using a Vaseline ring/physical barrier placed on the upper surface of the leaves. Plants were assessed for presence of mites and eggs on the scale described above five days after treatment. Results are shown in Table 8 below.

TABLE 8

| Treatment | Mites | Eggs |
|---|---|---|
| NRRL B-50550 WB 12.5% | 1.00 | 1.19 |
| NRRL B-50550 WB 6.25% | 1.51 | 1.73 |
| NRRL B-50550 WB 3.12% | 2.50 | 2.44 |
| NRRL B-50550 WB 1.56% | 2.12 | 2.19 |
| Positive Control AVID ® - abamectin | 1.46 | 1.30 |
| 0.8 μL/10 mL) | | |
| Untreated Control | 3.50 | 3.62 |

### Example 7

### Ovicidal Activity

NRRL B-50550 was tested for ovicidal activity as follows. Whole broth was prepared as described in Example 5. Two lima bean plants were preinfested with TSSM eggs by allowing adult female mites to oviposit on the leaf surface for 48 hours prior to treatment. Plants were then treated with 8 mL of various dilutions of whole broth. Plants were assessed five days after treatment. The number of live and dead eggs present in each treatment and control are shown in Table 9 below.

TABLE 9

| Treatment | Live Eggs | Dead Eggs |
|---|---|---|
| NRRL B-50550 (6.25%) | 1 | 32.75 |
| NRRL B-50550 (3.12%) | 0.5 | 18.25 |
| NRRL B-50550 (1.56%) | 1 | 20.5 |
| Positive Control | 1.5 | 75 |
| (OBERON ® SC - spiromesifen | | |
| 4 fl oz/100 gal) | | |
| Untreated Control | 24 | 2 |

### Example 8

### Drench Activity

Drench activity of NRRL B-50550 was studied using lima beans grown in sand. Whole broth was prepared as described in Example 3. Two applications of 10 mL each of a 12.5% dilution of whole broth were applied to the sand. Plants were watered carefully to prevent leaching of whole broth from the bottom of the pot. Applications were made at four days after planting and at five days after planting. Lower leaves were infested with motile TSSM three days after treatment two. The upper leaf trifoliate was infested nine days after lower leaves were infested. Assessments were made on lower leaves at 4, 5, 8 and 11 days after infestation. Assessments on upper leaves were conducted at two days after infestation. Results, based on the scoring system described in Example 2, are shown in Table 10 below.

TABLE 10

| | | | % Upper |
|---|---|---|---|
| | | | Leaf Surface |
| | Mites | Eggs | Stippled |
| NRRL B-50550 - 1st Assessment | 1.83 | 1.43 | 7 |
| [Lower Leaves] | | | |
| NRRL B-50550 - 2nd Assessment | 1.33 | 1.5 | 5 |
| [Lower Leaves] | | | |
| NRRL B-50550 - 3rd Assessment | 1.05 | 1.05 | 2.75 |
| [Lower Leaves] | | | |
| NRRL B-50550 - 4th Assessment | 1.83 | 1.38 | 4.5 |
| [Lower Leaves] | | | |
| NRRL B-50550 - 1st Assessment | 1.93 | 1.43 | 4.25 |
| [Upper Leaves] | | | |
| Untreated Control - 1st Assessment | 3.63 | 3.45 | 23.8 |
| [Lower Leaves] | | | |
| Untreated Control -2nd Assessment | 3.88 | 4 | 25 |
| [Lower Leaves] | | | |
| Untreated Control - 3rd Assessment | 4 | 4 | 52.5 |
| [Lower Leaves] | | | |
| Untreated Control - 4th Assessment | 4 | 4 | 80 |
| [Lower Leaves] | | | |
| Untreated Control - 1st Assessment | 4 | 4 | 77.5 |
| [Upper Leaves] | | | |

### Example 9

### Activity Against Fungal Phytopathogens

NRRL B-50550 was tested for activity against various plant fungal pathogens. It was found to be active against both wheat leaf rust and cucumber powdery mildew. Shake flasks containing Medium 1 were inoculated with frozen cultures of NRRL B-50550 and grown 1-2 days at 20-30° C. The resulting fermentation product was used to seed a 20 L bioreactor containing similar media and grown 1-2 days at 28° C. The resulting fermentation product was, in turn, used to seed a 200 L fermentor containing the following media: 7.0% starch, 3.0% dextrose, 1.5% yeast extract, 2.0% soy acid hydrolysate, 0.8% glycine, and 0.2% calcium carbonate. This medium was fermented at between 26° C. for 8 days. Six-day old wheat seedlings were treated with NRRL-50550 whole broth prepared at various dilutions in distilled water with 0.03% adjuvant (BREAK-THRU FIRST CHOICE® polyether-polymethylsiloxane-copolymer) shown in Table 11 below by covering both leaf surfaces with whole broth and allowing to dry. Seedlings were inoculated with a wheat leaf rust suspension one day after such treatment. Plants were rated about a week after treatment using the following scale on a 0-100% control, where 0% is no control and 100% is perfect control.

TABLE 11

| Treatment | Rate | Control |
|---|---|---|
| NRRL B-50550 WB | 20% | 98.7 |
| NRRL B-50550 WB | 5% | 95 |
| NRRL B-50550 WB | 1.25% | 50 |
| NRRL B-50550 WB | 0.31% | 0 |
| NRRL B-50550 Supernatant | 20% | 95 |
| NRRL B-50550 Supernatant | 5% | 66.7 |
| NRRL B-50550 Supernatant | 1.25% | 0 |
| NRRL B-50550 Supernatant | 0.31% | 0 |
| NRRL B -50550 Cell Extract | 20% | 50 |
| NRRL B-50550 Cell Extract | 5% | 50 |
| NRRL B-50550 Cell Extract | 1.25% | 0 |
| NRRL B-50550 Cell Extract | 0.31% | 0 |
| Untreated Check | | 0 |
| Adjuvant Check | | 0 |

In addition, NRRL B-50550 showed activity against cucumber powdery mildew when whole broth was applied on the lower leaf surface and the pathogen was applied on the upper leaf surface.

NRRL B-50550 also showed activity in a curative test against cucumber powdery mildew. Cucumber microplots were inoculated with cucumber powdery mildew at the point when plants had formed a dense canopy over the microplots and natural powdery mildew was just beginning to develop in adjacent plots. Six days post-infection, there was no visible evidence of disease from the inoculation. Freeze-dried powder of NRRL B-50550 was obtained from a fermentation broth prepared in a similar manner to that described in Example 13. Freeze-dried powder was then formulated with inert ingredients (a wetting agent, stabilizer, carrier, flow aid and dispersant) to make a wettable powder. The formulated product comprised 75% by weight freeze-dried powder. Wettable powder was diluted in water and applied at 100 gal/acre at the rates shown in Table 12, below. (Note that 100 gallons per acre translated to a spray volume of 200 mL per microplot.) Ratings were made on the same scale described above.

TABLE 12

| Plot | Treatment | Rating |
|---|---|---|
| NRRL B-50550 75 WP | 3.34 lb/A/100 gal | 95% |
| NRRL B-50550 75 WP | 1.67 lb/A/100 gal | 80% |
| NRRL B-50550 75 WP | 1.25 lb/A/100 gal | 80% |
| NRRL B-50550 75 WP | 0.83 lb/A/100 gal | 75% |
| Azoxystrobin, QUADRIS | 11 fl. oz./A/100 gal | 80% |
| Water check | | 0% |

### Example 10

### Corn Rootworm Activity

Tests were conducted to determine efficacy of NRRL B-50550 against corn rootworm. NRRL B-50550 whole broth was prepared in Medium 1 or Medium 2, as described in Example 2. NRRL B-50550 whole broth was diluted and fed to larvae of western spotted cucumber beetle (Diabrotica undecimpunctata) in a diet-based assay conducted in a microtiter plate. Activity was assessed and rated on a scale of 1 to 4, as described in Example 2. The termiticide/insecticide TERMIDOR® SC (5-amino-1-(2,6-dichloro-4(trifluoromethyl)phenyl)-4-((1,R,S)-(trifluoromethyl)sulfinyl)-1-H-pyrazole-3-carbonitrile, commonly known as fipronil BASF) was used as positive control. Results are shown in Table 13. NRRL B-50550 showed the same insecticidal activity as the insecticide TERMIDOR® SC, which contains the active ingredient fipronil.

TABLE 13

| Treatment | Dosage | Rating |
|---|---|---|
| NRRL B-50550 Media 1 | 100% | 1 |
| NRRL B-50550 Media 1 | 25% | 1 |
| NRRL B-50550 Media 1 | 6.25% | 1 |
| NRRL B-50550 Media 1 | 1.56% | 3.75 |
| NRRL B-50550 Media 2 | 100% | 1 |
| NRRL B-50550 Media 2 | 25% | 1 |
| NRRL B-50550 Media 2 | 6.25% | 1 |
| NRRL B-50550 Media 2 | 1.56% | 4 |
| TERMIDOR ® SC 8.3 mg/mL | 100.00% | 1 |
| TERMIDOR ® SC | 25.00% | 1 |
| TERMIDOR ® SC | 1.56% | 3.75 |
| Untreated | | 4 |

### Example 11

### Dose/Response Laboratory Assay

A study was conducted to determine the response of TSSM to different doses of NRRL B-50550. Whole broth was prepared as described in Example 5. The resulting whole broth was diluted to the percentages shown in Table 14 below using water and 0.35% surfactant. Water and 0.35% surfactant were used as the control treatment. In two separate trials, the whole broth solutions and a control treatment were applied to run-off to the lower surface of lima bean leaves, with four replicates per dose. Plants were infested one day after such treatment with 50-100 TSSM, and assessed for the presence of mites and eggs on the scale described above five days after treatment. Results are shown in Table 14 below.

TABLE 14

| Percent Whole Broth | Mite Rating | Mortality |
|---|---|---|
| 0.20 | 3.55 | 15% |
| 0.39 | 3.17 | 25% |
| 0.78 | 2.11 | 70% |
| 1.57 | 1.52 | 90% |
| 3.13 | 1.22 | 95% |

At the lowest concentration tested (0.20% whole broth), significant mortality was observed based on the error bars of the treatment compared to the control treatment. It was observed that part of the effect associated with application of NRRL B-50550 is that it causes mites to leave the plant. Thus, even at sublethal doses NRRL B-50550 may reduce the mite population on a plant.

### Example 12

### Activity Against Abamectin-Resistant Spider Mites

A study was performed to determine the activity of NRRL B-50550 against abamectin-resistant spider mites (Tetranychus urticae strain NL), as compared to wild-type spider mites (Tetranychus urticae strain RW). French bean plants were treated with a wettable powder of a fermentation product of NRRL B-50550 prepared as described in the last paragraph of Example 9, at the rates shown in Table 15 below after dilution. Plants were infested one day prior to treatment with 50-100 of either strain NL or RW, and assessed for the presence of mites seven and fourteen days after treatment. Results are shown in Table 15 below.

TABLE 15

| | | 7 days | 14 days | 7 days | 14 days |
|---|---|---|---|---|---|
| | Dos- | Resistant | Resistant | Wild Type | Wild Type |
| | age | Mites (% | Mites (% | Mites (% | Mites (% |
| Treatment | (ppm) | control) | control) | control) | control) |
| NRRL | 100 | 95 | 95 | 80 | 90 |
| B-50550 | | | | | |
| 75 WP | | | | | |
| NRRL | 20 | 50 | 50 | 30 | 30 |
| B-50550 | | | | | |
| 75 WP | | | | | |
| NRRL | 4 | 0 | 0 | 0 | 0 |
| B-50550 | | | | | |
| 75 WP | | | | | |
| Abamectin | 20 | 99 | 99 | 100 | 100 |
| Abamectin | 4 | 99 | 80 | 100 | 100 |
| Abamectin | 0.8 | 80 | 0 | 100 | 100 |
| Abamectin | 0.16 | 0 | 0 | 99 | 100 |
| Water | | 0 | 0 | 0 | 0 |

### Example 13

### Fermentation Product Containing Increased Levels of Gougerotin—Use of Glycine

Fermentation was conducted to optimize gougerotin production and miticidal activity of NRRL B-50550. A primary seed culture was prepared as described in Example 1 using a media composed of 10.0 g/L starch, 15.0 g/L glucose, 10.0 g/L yeast extract, 10.0 g/L casein hydrolysate (or 10.0 g/L soy peptone) and 2.0 g/L CaCO₃ in 2 L shake flasks at 20-30° C. When there was abundant mycelial growth in the shake flasks, after about 1-2 days, the contents were transferred to fresh media (same as above, with 0.1% antifoam) and grown in a 400 L fermentor at 20-30° C. When there was abundant mycelial growth, after about 20-30 hours, the contents were transferred to a 3000 L fermentor and grown for 160-200 hours at 20-30° C. in media composed of 80.0 g/L (8.0%) Maltodextrin, 30.0 g/L (3.0%) glucose, 15.0 g/L (1.5%) yeast extract, 20.0 g/L (2.0%) soy acid hydrolysate, 10.0 g/L (1.0%) glycine and 2.0 g/L (0.2%) calcium carbonate and 2.0 ml/L antifoam.

TABLE 16
Yield and Normalized Gougerotin Productivity

| | | | | | Normalized |
|---|---|---|---|---|---|
| | Harvest | Harvest | Total | Target | Volumetric |
| | Titer | Weight | Gougerotin | Volume | Titer |
| | (mg/g) | (kg) | (kg) | (L) | (g/L) |
| First 3000 L | 1.7 | 3397 | 5.78 | 3000 | 1.9 |
| Fermentation | | | | | |
| Second 3000 L | 1.8 | 3511 | 6.33 | 3000 | 2.1 |
| Fermentation | | | | | |

Using the first 3000 L fermentation as an example, the yield of gougerotin in the fermentor is calculated as follows. 3397 kg×1.7 mg/g Fermentation broth=5774.90 g gougerotin=5.78 kg. The initial weight in the fermentor was 3496 kg (3256 kg Medium+240 kg Seed), which resulted in a final volume more than the target volume 3000 L. Since the target volume 3000 L is the basis for calculating the amount of all ingredients in the production medium, the normalized volumetric productivity is: 5774.9 g/3000 L=1.9 g/L (see Table 16). This gougerotin concentration was similar to the 1.8 g/L achieved in a 20 L fermentation conducted using the same media as described above, with the final fermentation step and media containing glycine (as amino acid).

Throughout this application, gougerotin levels are detected using analytical HPLC chromatography as described in Example 15 below.

### Example 14

### Fermentation Product Containing Increased Levels of Gougerotin—Use of Glutamic Acid

Fermentation was conducted to optimize gougerotin production and miticidal activity of NRRL No. B-50550. A primary seed culture was prepared as described in Example 1 using a media composed of 10.0 g/L starch, 15.0 g/L glucose, 10.0 g/L yeast extract, 10.0 g/L casein hydrolysate (or 10.0 g/L soy peptone) and 2.0 g/L CaCO₃ in 1 L shake flasks at 20-30° C. When there was abundant mycelial growth in the shake flasks, after about 1-2 days, the contents were transferred to fresh media (same as above, with 0.1% antifoam) and grown in 1 L shake flasks at 20-30° C. When there was abundant mycelial growth, after about 20-30 hours, the contents were transferred to a 20 L fermentor and grown for 160-200 hours at 20-30° C. in media composed of 60.0 g/L (8.0%) starch, 30.0 g/L (3.0%) dextrose, 15.0 g/L (1.5%) yeast extract, 20.0 g/L (2.0%) soy acid hydrolysate, 12.0 g/L (1.0%) L-glutamic acid and 2.0 g/L (0.2%) calcium carbonate and 2.0 mL/L antifoam.

This gougerotin concentration using L-glutamic acid as amino acid in this fermentation was 1.1 g/L.

### Example 15

### Gougerotin-Overproducing Mutants

With the goal of increasing gougerotin production and bioactivity, mutants were created from the parent strain Streptomyces microflavus NRRL No. B-50550 through an antibiotic-resistant mutant screening program in which libraries of mutants resistant to individual antibiotics (gentamicin, rifampicin, streptomycin, paromomycin or tobramycin) were produced. See, Okamoto-Hosoya, Y., et al., The Journal of Antibiotics 43(12) December 2000. The parent strain was subjected to mutagenesis using N-methyl-N′-nitro-N-nitrosoguanidine (“NTG”) and then resulting antibiotic resistant mutants selected and screened. A detailed description of creation and screening of mutant libraries from which gougerotin-overproducing strains were selected for further development is described below.

Spore suspensions of Streptomyces microflavus NRRL No. B-50550 were prepared from soy flour maltose (SFM) agar plates containing B-50550 grown for approximately 14 days or to sporulation and stored at −80° C. in 20% glycerol. NTG, dissolved in suitable buffer, was added to the spore suspensions in an amount suitable to obtain at least 50% kill (e.g., 75%-95% kill) (0.5 mg/mL at pH 8.5 slowly shaken for 1 hour at 37° C.). NTG-treated spore suspensions were then plated onto GYM (glucose 4 g/L, yeast extract 4 g/L, malt extract 10 g/L, and agar 12 g/L) supplemented with the following concentrations of antibiotics. See Table 17 below.

TABLE 17

| ANTIBIOTIC | 1x | | 2x | | 5x | | 10x | 20x | |
|---|---|---|---|---|---|---|---|---|---|
| Streptomycin SO₄ | 10 | mg/L | 20 | mg/L | 50 | mg/L | 100 mg/L | 200 | mg/L |
| Rifampicin (Fresh) | 3.5 | mg/L | 7 | mg/L | 17.5 | mg/L | 35 mg/L | 70 | mg/L |
| Paromomycin SO₄ | 1 | mg/L | 2 | mg/L | 5 | mg/L | 10 mg/L | 20 | mg/L |
| Tobramycin SO₄ | 4.5 | mg/L | 9 | mg/L | 22.5 | mg/L | 45 mg/L | 90 | mg/L |
| Gentamycin SO₄ | 5.5 | mg/L | 11 | mg/L | 27.5 | mg/L | 55 mg/L | 110 | mg/L |

See Kieser, T., et al., Practical Streptomyces Genetics, Ch. 5 John Ines Centre Norwich Research Park, England (2000), pp. 99-107. Approximately 350 individual antibiotic-resistant colonies were isolated, purified, and screened as described below.

Each isolate removed from GYM antibiotic plates was re-plated onto SFM agar plates. Agar plugs containing antibiotic-resistant bacteria or antibiotic-resistant bacterial colonies picked using an instrument or by hand using pipette tips were used to inoculate 24-well blocks containing about 2.5 to 3.5 mL of seed media. Bacteria in these inoculated blocks were grown for 3 days and the resulting culture broth used to inoculate 24-well blocks containing production media. Bacteria in production blocks were grown for seven days at 28° C. with agitation. Each well in the seed blocks contained Trypticase Soy Broth (TSB) (Per liter of DI H₂0: 17 g Bacto Tryptone (Pancreatic Digest of Casein), 3 g Bacto Soytone (Pancreatic Digest of Soybean Meal), 2.5 g Dextrose, 5 g NaCl, 2.5 g Dipotassium Phosphate) and in the production blocks contained Medium 2 of Example 2 (Proflo 20 g/L, malt extract 20 g/L, KH₂PO₄ monobasic 6 g/L, K₂HPO₄ dibasic 4.8 g/L).

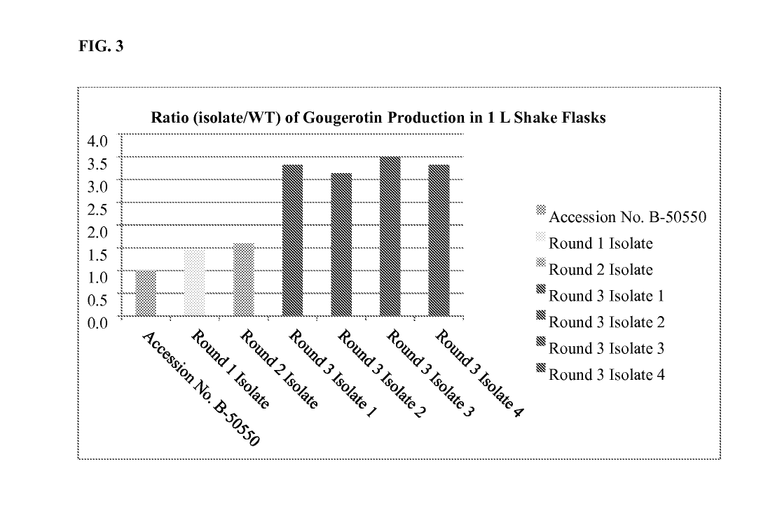

The whole broth from each well of the production block was tested for gougerotin production as follows using analytical HPLC chromatography. 2.4 mL water was added to each well of the production block. Blocks were vortexed and centrifuged. 0.8 mL supernatant was transferred to an extraction block containing 4 mL of water per well. 3.2 mL water was added to the cell pellet in each well of the production block and the block vortexed and centrifuged again. This 3.2 mL of wash water was then added to the appropriate well of each extraction block. The aqueous extracts in the extraction block were then assayed for gougerotin content using analytical HPLC chromatography. Specifically, a sample was injected onto a Cogent Diamond hydride column (100 A, 4 μm, 150×4 6 mm) fitted with a Diamond Hydride guard column. The column was eluted with a 30 minute Acetonitrile/NH₄OAC gradient (see below). The flow rate was 1 mL/min Gougerotin was detected at 254 nm Gougerotin elutes as a single peak with an approximate retention time of 19 minutes. Certain top overproducing mutants were confirmed by re-growing in both 24 well blocks and 250 mL flasks to confirm gougerotin levels. Once confirmed some isolates were then subjected to at least one more round of mutagenesis (i.e., mutagenic event) and antibiotic-resistance screening. Each subsequent round of mutagenesis coupled with antibiotic-screening was performed using the remaining antibiotics to which an isolate derived in the previous round had not developed resistance. Small (1.2×) increases in gougerotin production were found after a single round of screening, and subsequent rounds lead to greater increases from isolates generated from the same original low level overproducer, which produced about 0.3 mg/g gougerotin when cultured on a small scale using basic media in these studies. See FIG. 3.

Selected mutants with higher gougerotin production and a general ability to sporulate on SFM agar plates were grown in 1 L baffled shake flasks and subsequently scaled up to 5 L Sartorius B-plus bioreactors and/or 20 L bioreactors containing Medium 2. See FiI 4.

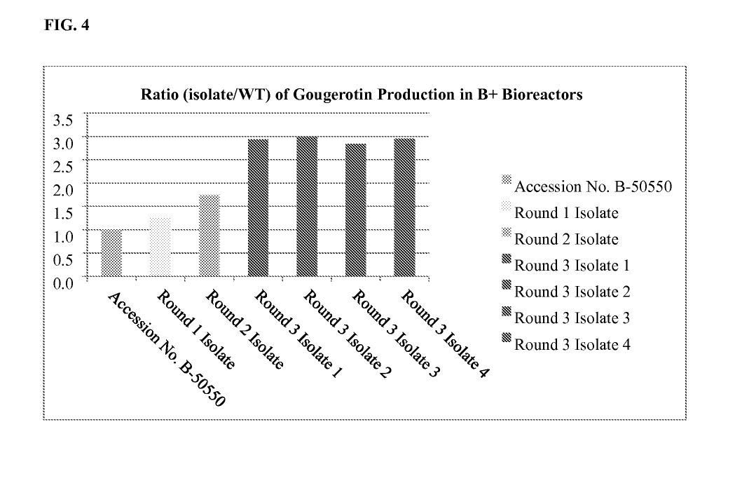

The strain designated as Round 3 Isolate 4 in FIGS. 3 and 4 was selected for scale-up according to the process described in Example 13. This strain produced a fermentation broth containing 3.8 mg/g of gougerotin.

### Example 16

### Conversion Rate: Whole Broth to Freeze-Dried Powder

Table 18 shows the conversion rate between whole broth to freeze-dried powder for several lots of whole broth of B-50550 prepared as described in Example 13. These calculations assume that whole broth is converted completely to freeze-dried powder and a density of whole broth of 1 g/mL. (Note that density of fermentation broths before any downstream processing is about 1 g/mL.) The “average %” is the average percentage by weight of freeze dried powder obtained from a certain lot of whole broth.

TABLE 18

| Lot of | | Kg dry | Lbs Dry Weight | Gouge- | Gouge- |
|---|---|---|---|---|---|
| Whole | | Weight | Freeze-Dried | rotin | rotin |
| Broth | Aver- | per | Powder (“FDP”) | (mg/g) | (mg/g) |
| (“WB”) | age % | Gallon | Per Gallon | WB | FDP |
| A | 5.93% | 224.47422 | 0.49488135 | 1.7 | 28.7 |
| B | 7.08% | 268.00632 | 0.59085328 | 1.5 | 21.2 |

### Example 17

### Genome Shuffling to Identify Gougerotin-Overproducing Mutants

Mutants were isolated with genome shuffling screens using the following protocol. A genome shuffling pool was comprised of Streptomyces microflavus strains producing gougerotin generated with chemical mutagenesis of Streptomyces microflavus strain NRRL B-50550 as described in Example 15. Included in this pool was Streptomyces microflavus strain Strain No. 091013-02 (also known as Streptomyces microflavus strain M, AQ6121.002, and AQ32392). Individual members of the genome shuffling pool were cultured at 220 RPM, 28° C. for approximately 40 hrs in 250 mL shake flasks containing 50 mLs of a culture medium containing 3 g/L yeast extract, 5 g/L peptone, 3 g/L malt extract, 10 g/L glucose, 150 g/L sucrose, 5 mM MgCl₂, and 0.5% glycine. Following pelleting by centrifugation, cells were resuspended in water (a sucrose solution was subsequently added to the cell suspension). Pelleting and resuspension were performed twice. Cells were incubated for 15-45 minutes in 2 mg/mL egg white lysozyme in Protoplast “P” Buffer (103 g/L sucrose; 0.25 g/L K₂SO₄; 2.02 g/L MgCl₂.6H₂O; 0.005% KH₂PO₄; 0.3% CaCl₂; 0.6% TES, pH 7.2; and trace amounts of ZnCl₂, FeCl₃.6H₂O, CuCl₂.2H₂O, MnCl₂.4H₂O, Na₂B₄O₇.10H₂O, and (NH₄)₂Mo₇O₂₄.4H₂O). Vegetative cell to protoplast conversion was monitored under a phase contrast microscope. Any non-protoplast cells were removed by filtration through sterile cheesecloth and protoplast suspensions were prepared by dilution with a sterile, buffered solution (e.g., P Buffer). Protoplast solutions for each variant were combined in a PEG solution and incubated at room temperature. Following incubation, protoplast suspensions were plated onto agar plates containing 103 g/L sucrose; 0.25 g/L K₂SO₄; 10.12 g/L MgCl₂.6H₂O; 10 g/L glucose; 0.1 g/L casamino acids; 5 g/L yeast extract; 22 g/L agar; 0.005% KH₂PO₄; 0.3% CaCl₂.2H₂O; 0.3% L-proline; 0.6% TES, pH 7.2; and trace amounts of ZnCl₂, FeCl₃.6H₂O, CuCl₂.2H₂O, MnCl₂.4H₂O, Na₂B₄O₇.10H₂O, and (NH₄)₂Mo₇O₂₄.4H₂O) and incubated at 30° C. for 72 hours.

Screening of genome shuffled strains proceeded as follows. Individual bacterial colonies were evaluated for gougerotin production as described above in Example 15 except that no antibiotic was present in the media. Several gougerotin-overproducing mutants were identified from the genome shuffling screens. Among these gougerotin-overproducing mutants were the five strains identified in Table 19.

TABLE 19

| Species | NRRL Accession Number |
|---|---|
| Streptomyces microflavus | NRRL B-50954 |
| Streptomyces microflavus | NRRL B-50955 |
| Streptomyces microflavus | NRRL B-50956 |
| Streptomyces microflavus | NRRL B-50957 |
| Streptomyces microflavus | NRRL B-50958 |

### Example 18

### Quantification of Gougerotin in Genome Shuffled Strains

Each of the strains identified in Example 17 were cultured in TSB and then in Medium 2 or Medium 2 supplemented with glycine at a concentration similar to that used in Examples 13 (designated “Medium 2+Glycine”). Samples from each of the whole broths from the cultures were centrifuged and the supernatants filtered. The filtered supernatants were then analyzed for gougerotin concentrations with analytical HPLC chromatography as described in Example 15.

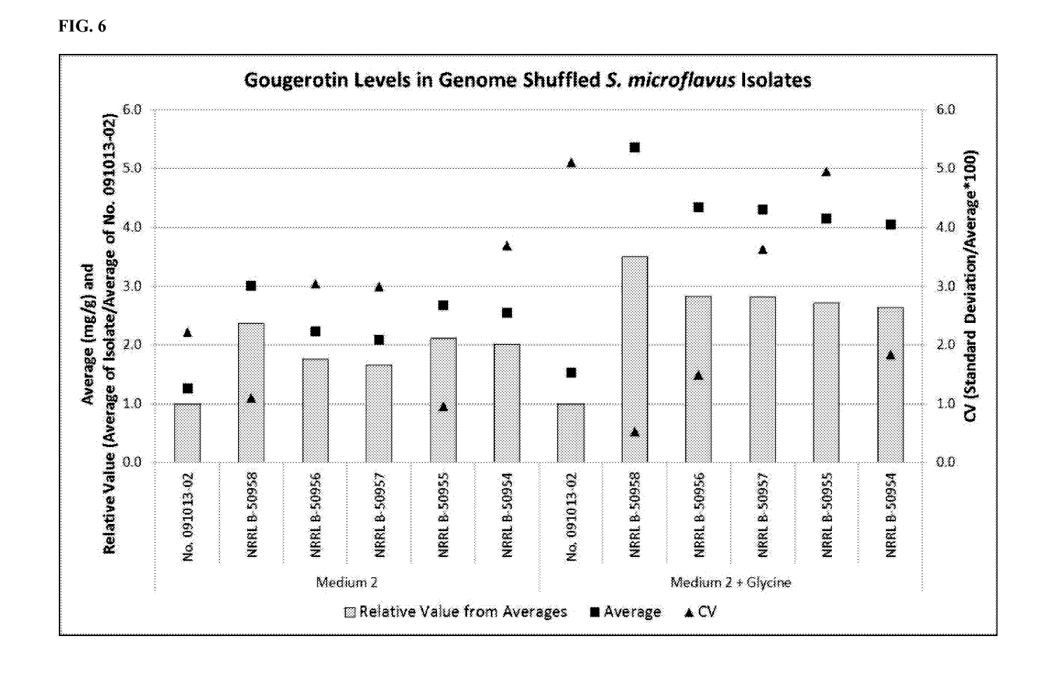

FIG. 6 presents the results of the analytical HPLC chromatography. Each sample was analyzed with four HPLC replicates, and the average gougerotin concentration (indicated as mg gougerotin/g whole broth supernatant or mg/g) is shown (▪). Also shown are the coefficients of variation (CVs) for each average (▴). Each of the five strains produced a whole broth supernatant with an average gougerotin concentration that was at least 2 mg/g. Because the density of the whole broth supernatant was slightly greater than 1 g/mL, the average concentration of gougerotin in each of the whole broth supernatants was at least 2 mg/mL (i.e., at least 2 g/L).

The relative values for each of the five strains were calculated as the average gougerotin concentration of an isolate divided by the average gougerotin concentration of Streptomyces microflavus strain No. 091013-02 (also known as Streptomyces microflavus strain M, AQ6121.002, and AQ32392) grown in the same culture medium. Streptomyces microflavus strain No. 091013-02 was one of the strains included in the initial genome shuffling pool. The relative values are shown as the bars in the graph of FIG. 6. The genome shuffled strains produced about twice as much gougerotin as Streptomyces microflavus strain No. 091013-02 when cultured in Medium 2 and almost three times as much gougerotin as Streptomyces microflavus strain No. 091013-02 when cultured in Medium 2+Glycine.

In a separate experiment to quantify gougerotin in a genome shuffled strain generated in Example 17, the strain was cultured in medium containing glycine as described in Example 13. Using a 3000 L fermentation, the yield of gougerotin in the fermentor was calculated as follows. 3302 kg×5.0 mg/g fermentation broth=16510 g gougerotin=16.51 kg. The initial weight in the fermentor was 3206 kg (3013 kg Medium+193 kg Seed), which resulted in a final volume more than the target volume 3000 L. Since the target volume 3000 L is the basis for calculating the amount of all ingredients in the production medium, the normalized volumetric productivity is: 16510 g/3000 L=5.5 g/L (see Table 20).

TABLE 20
Yield and Normalized Gougerotin Productivity

| | | | | | Normalized |
|---|---|---|---|---|---|
| | Harvest | Harvest | Total | Target | Volumetric |
| | Titer | Weight | Gougerotin | Volume | Titer |
| | (mg/g) | (kg) | (kg) | (L) | (g/L) |
| 3000 L | 5.0 | 3302 | 16.51 | 3000 | 5.5 |
| Fermentation | | | | | |

Table 21 shows the conversion rate between whole broth to freeze-dried powder for one lot of whole broth of a genome shuffled strain prepared as described in Example 13. These calculations assume that whole broth is converted completely to freeze-dried powder and a density of whole broth of 1 g/mL. (Note that density of fermentation broths before any downstream processing is about 1 g/mL.) The “average %” is the average percentage by weight of freeze dried powder obtained from a certain lot of whole broth.

TABLE 21

| Lot of | | Kg dry | lbs dry weight | Gouge- | Gouge- |
|---|---|---|---|---|---|
| Whole | | weight | freeze-dried | rotin | rotin |
| Broth | Aver- | per | powder (“FDP”) | (mg/g) | (mg/g) |
| (“WB”) | age % | gallon | per gallon | WB | FDP |
| C | 5.86% | 261.26467 | 0.575989322 | 5.1 | 73.91 |

### Example 19

### Activity Against Spider Mites of Genome Shuffled Strains

Each of the five Streptomyces microflavus mutant strains identified in Example 17 and Streptomyces microflavus strain No. 091013-02 was cultured in Medium 2 or in Medium 2+Glycine, and the resulting fermentation products were evaluated for activity against spider mites as described in Example 2. The results are shown in Table 21 below with a lower numeric rating indicating increased mortality as explained in Example 2. All five of the fermentation products from the genome shuffled strains showed superior control of spider mites compared to the fermentation product from Streptomyces microflavus strain No. 091013-02. This difference in spider mite control between the genome shuffled strains and Streptomyces microflavus strain No. 091013-02 was more pronounced in the fermentation products generated with Medium 2+Glycine than in those generated with Medium 2.

TABLE 21

| Fermentation Product | Mites | Eggs |
|---|---|---|
| Untreated Control | 3.2 | 2.5 |
| No. 091013-02 in Medium 2 | 3.5 | 2.5 |
| NRRL B-50958 in Medium 2 | 1.2 | 1.1 |
| NRRL B-50956 in Medium 2 | 1.9 | 2.1 |
| NRRL B-50957 in Medium 2 | 2.1 | 2.3 |
| NRRL B-50955 in Medium 2 | 2.2 | 1.7 |
| NRRL B-50954 in Medium 2 | 2.2 | 2.3 |
| Untreated Control | 3.4 | 3.1 |
| No. 091013-02 in Medium 2 + Glycine | 2.5 | 2.4 |
| NRRL B-50958 in Medium 2 + Glycine | 1.1 | 1.1 |
| NRRL B-50956 in Medium 2 + Glycine | 1.2 | 1.1 |
| NRRL B-50957 in Medium 2 + Glycine | 1.6 | 1.8 |
| NRRL B-50955 in Medium 2 + Glycine | 1.8 | 2.0 |
| NRRL B-50954 in Medium 2 + Glycine | 1.5 | 1.4 |

### Example 20

### Genome Shuffled Strains' Curative Activity Against Powdery Mildew of Cucurbits

Streptomyces microflavus strain No. 091013-02, Streptomyces microflavus strain NRRL B-50958, and several other genome shuffled strains generated in Example 17 were cultured to produce fermentation broths that were formulated as wettable powders generally following the procedure outlined in Example 13 with minor modifications such as decreased fermentor volumes. The average gougerotin concentration measured in the Streptomyces microflavus strain 091013-02 fermentation broth was 3.4 mg/mL while the average gougerotin concentration of the fermentation broth from the genome shuffled strains was 5.2-5.4 mg/mL. The wettable powders from the fermentation broths were diluted in distilled water containing 0.03% of a nonionic surfactant to final dilutions of 4%, 2%, 1%, 0.50% and 0.25% prior to application to plants.

Young cucumber plants were exposed to a fungal inoculum containing Podosphaera xanthii (Powdery Mildew of Cucurbits). A few days later, the infected cucumber plants were treated with each of the diluted wettable powders at the specified rates. Untreated control plants and plants treated with QUADRIS® (azoxystrobin) at 25 ppm were included for purposes of comparison. Several days after treatment, each plant was scored for percent control of the pathogen relative to the untreated control plants. Each treatment was evaluated with three replicates and the average percent control reported (see Table 22).

TABLE 22

| | Application | Curative Activity |
|---|---|---|
| Treatment | Rate | (% Control) |
| Strain 091013-02 | 4% | 95 |
| | 2% | 92 |
| | 1% | 80 |
| | 0.50% | 67 |
| | 0.25% | 33 |
| Strain NRRL B-50958 | 4% | 99 |
| | 2% | 93 |
| | 1% | 87 |
| | 0.50% | 75 |
| | 0.25% | 67 |
| QUADRIS ® (azoxystrobin) | 25 ppm | 82 |
| Untreated | | 0 |

Streptomyces microflavus strain NRRL B-50958 demonstrated greater curative activity against Podosphaera xanthii (Powdery Mildew of Cucurbits) than did the parental strain, Streptomyces microflavus strain 091013-02. The other genome shuffled strains had curative activity against Podosphaera xanthii (Powdery Mildew of Cucurbits) similar to that of Streptomyces microflavus strain NRRL B-50958. These differences in antifungal activity were more pronounced at the lower application rates.

### Example 21

### Genome Shuffled Strains' Preventative Activity Against Powdery Mildew of Cucurbits

Wettable powders were prepared for Streptomyces microflavus strain 091013-02 and several of the genome shuffled strains generated in Example 17 as described in Example 20. The wettable powders from the fermentation broths were diluted in distilled water containing 0.03% of a nonionic surfactant to final dilutions of 4%, 2%, 1%, 0.50% and 0.25% prior to application to plants.

Young cucumber plants were treated with each of the diluted wettable powders at the specified rates. Untreated control plants and plants treated with QUADRIS® (azoxystrobin) at 25 ppm were included for purposes of comparison. Subsequently, the plants were exposed to a fungal inoculum containing Podosphaera xanthii (Powdery Mildew of Cucurbits). Several days after inoculation with the Powdery Mildew, each plant was scored for percent control of the pathogen relative to the untreated control plants. Each treatment was evaluated with three replicates and the average percent control reported (see Table 23). The other genome shuffled strains had preventative activity against Podosphaera xanthii (Powdery Mildew of Cucurbits) similar to that of Streptomyces microflavus strain NRRL B-50958.

TABLE 23

| | Application | Preventative Activity |
|---|---|---|
| Treatment | Rate | (% Control) |
| Strain 091013-02 | 4% | 90 |
| | 2% | 78 |
| | 1% | 75 |
| | 0.50% | 17 |
| | 0.25% | 0 |
| Strain NRRL B-50958 | 4% | 95 |
| | 2% | 88 |
| | 1% | 67 |
| | 0.50% | 0 |
| | 0.25% | 0 |
| QUADRIS ® (azoxystrobin) | 25 ppm | 95 |
| Untreated | | 0 |

### Example 22

### Efficacy of Streptomyces microflavus Mutant Strain in Apple Tree Field Trials with Apple Scab (Venturia inaequalis)

Two field trials were conducted with a fermentation product of Streptomyces microflavus strain No. 091013-02 formulated as a suspension concentrate on apple trees, naturally infected with the causal agent of apple scab, Venturia inaequalis. Ten treatments with an application volume of 1000 L/ha at 2 m cph were done between April 23 and June 30 at a growth stage of BBCH62 to BBCH77 in 5 to 11 days interval as outlined in Table 25. The percent disease control shown in Table 24 is the result of the last evaluation made 11 days after the final application, done by visual observation of disease symptoms. 0% means an efficacy which corresponds to that of the untreated control while an efficacy of 100% means that no disease was observed.

TABLE 24

| | | | Disease Control |
|---|---|---|---|
| | Dosage | Application | in % Mean of |
| Treatment | L/ha | Code | 2 Trials |
| Untreated Control | | | 0 |
| Streptomyces microflavus | 2 | ABCDEFGHIJ | 67 |
| strain No. 091013-02 | | | |
| (suspension concentrate) | | | |
| Streptomyces microflavus | 1 | ABCDEFGHIJ | 57 |
| strain No. 091013-02 | | | |
| (suspension concentrate) | | | |

TABLE 25

| Application | Application | Growth |
|---|---|---|
| Code | Date | Stage |
| A | April 23 | 62 |
| B | April 28 | 63 |
| C | May 5 | 67 |
| D | May 12 | 71 |
| E | May 19 | 72 |
| F | May 26 | 73 |
| G | June 2 | 74 |
| H | June 11 | 75 |
| I | June 19 | 76 |
| J | June 30 | 77 |

### Example 23

### Efficacy of Streptomyces microflavus Mutant Strain in Grapevine Field Trials with Powdery Mildew (Uncinula necator)

Two field trials were conducted with a fermentation product of Streptomyces microflavus strain No. 091013-02 formulated as a suspension concentrate on grapevine, naturally infected with Uncinula necator. Six treatments with an application volume of 1000 L/ha were done between June 3 and July 1 at a growth stage of BBCH57 to BBCH75 in 5 to 7 days interval as outlined in Table 27. The percent disease control shown in Table 26 is the result of the last evaluation made 15 days after the final application, done by visual observation of disease symptoms. 0% means an efficacy which corresponds to that of the untreated control while an efficacy of 100% means that no disease was observed.

TABLE 26

| | | | Disease Control |
|---|---|---|---|
| | Dosage | Application | in % Mean of |
| Treatment | L/ha | Code | 2 Trials |
| Untreated Control | | | 0 |
| Streptomyces microflavus | 2 | ABCDEF | 100 |
| strain No. 091013-02 | | | |
| (suspension concentrate) | | | |
| Streptomyces microflavus | 1 | ABCDEF | 99 |
| strain No. 091013-02 | | | |
| (suspension concentrate) | | | |

TABLE 27

| Application | Application | Growth |
|---|---|---|
| Code | Date | Stage |
| A | June 3 | 57 |
| B | June 10 | 60 |
| C | June 16 | 64 |
| D | June 21 | 71 |
| E | June 26 | 73 |
| F | July 1 | 75 |

### Example 24

### Efficacy of Streptomyces microflavus Mutant Strain in Zucchini Field Trials with Powdery Mildew (Sphaerotheca fuliginea)

Two field trials were conducted with a fermentation product of Streptomyces microflavus strain No. 091013-02 formulated as a suspension concentrate on zucchini, artificially inoculated with Sphaerotheca fuliginea. Five treatments with an application volume of 1000 L/ha were done between July 15 and August 8 at a growth stage of BBCH59 to BBCH72 in 4 to 8 days interval as outlined in Table 29. The percent disease control shown in Table 28 is the result of the last evaluation made 10 days after the final application, done by visual observation of disease symptoms. 0% means an efficacy which corresponds to that of the untreated control while an efficacy of 100% means that no disease was observed.

TABLE 28

| | | | Disease Control |
|---|---|---|---|
| | Dosage | Application | in % Mean of |
| Treatment | L/ha | Code | 2 Trials |
| Untreated Control | | | 0 |
| Streptomyces microflavus | 2 | ABCDE | 100 |
| strain No. 091013-02 | | | |
| (suspension concentrate) | | | |
| Streptomyces microflavus | 1 | ABCDE | 89 |
| strain No. 091013-02 | | | |
| (suspension concentrate) | | | |

TABLE 29

| Application | Application | Growth |
|---|---|---|
| Code | Date | Stage |
| A | July 15 | 59 |
| B | July 23 | 65 |
| C | July 30 | 71 |
| D | August 4 | 72 |
| E | August 8 | 72 |

Unless defined otherwise, all technical and scientific terms herein have the same meaning as commonly understood by one of ordinary skill in the art to which this invention belongs. All publications, patents, and patent publications cited are incorporated by reference herein in their entirety for all purposes.

It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

We claim:

1. A composition comprising a biologically pure culture of a strain selected from the group consisting of Streptomyces microflavus strain NRRL B-50954, Streptomyces microflavus strain NRRL B-50955, Streptomyces microflavus strain NRRL B-50956, Streptomyces microflavus strain NRRL B-50957, Streptomyces microflavus strain NRRL B-50958, and mutants thereof having all the identifying characteristics of the respective strain.

2. The composition of claim 1, wherein the composition is a fermentation product of the strain.

3. The composition of claim 1, wherein the strain is Streptomyces microflavus strain NRRL B-50958 or a mutant thereof having all the identifying characteristics of the strain.

4. The composition of claim 1 comprising at least about 1×10⁶ CFU of the strain/mL culture.

5. The composition according to any one of the preceding claims further comprising a formulation ingredient.

6. The composition of claim 5, wherein the formulation ingredient is a wetting agent.

7. The composition of claim 1 formulated as a water-dispersible granule or a wettable powder.

8. A method of treating a plant to control a plant disease or pest, wherein the method comprises applying a composition comprising a strain selected from the group consisting of Streptomyces microflavus strain NRRL B-50954, Streptomyces microflavus strain NRRL B-50955, Streptomyces microflavus strain NRRL B-50956, Streptomyces microflavus strain NRRL B-50957, Streptomyces microflavus strain NRRL B-50958, and mutants thereof having all the identifying characteristics of the respective strain, to the plant, to a part of the plant and/or to a locus of the plant.

9. The method of claim 8, wherein the composition is a fermentation product of the strain.

10. The method of claim 8, wherein the strain is Streptomyces microflavus strain NRRL B-50958 or a mutant thereof having all the identifying characteristics of the strain.

11. The method of claim 8, wherein the method comprises applying the composition to foliar plant parts.

12. The method of claim 8, wherein the pest to be controlled is selected from a mite and Diabrotica spp.

13. The method of claim 12, wherein the mite is selected from the group consisting of clover mite, brown mite, hazelnut spider mite, asparagus spider mite, brown wheat mite, legume mite, oxalis mite, boxwood mite, Texas citrus mite, Oriental red mite, citrus red mite, European red mite, yellow spider mite, fig spider mite, Lewis spider mite, six-spotted spider mite, Willamette mite Yuma spider mite, web-spinning mite, pineapple mite, citrus green mite, honey-locust spider mite, tea red spider mite, southern red mite, avocado brown mite, spruce spider mite, avocado red mite, Banks grass mite, carmine spider mite, desert spider mite, vegetable spider mite, tumid spider mite, strawberry spider mite, two-spotted spider mite, McDaniel mite, Pacific spider mite, hawthorn spider mite, four-spotted spider mite, Schoenei spider mite, Chilean false spider mite, citrus flat mite, privet mite, flat scarlet mite, white-tailed mite, pineapple tarsonemid mite, West Indian sugar cane mite, bulb scale mite, cyclamen mite, broad mite, winter grain mite, red-legged earth mite, filbert big-bud mite, grape erineum mite, pear blister leaf mite, apple leaf edgeroller mite, peach mosaic vector mite, alder bead gall mite, Perian walnut leaf gall mite, pecan leaf edgeroll mite, fig bud mite, olive bud mite, citrus bud mite, litchi erineum mite, wheat curl mite, coconut flower and nut mite, sugar cane blister mite, buffalo grass mite, bermuda grass mite, carrot bud mite, sweet potato leaf gall mite, pomegranate leaf curl mite, ash sprangle gall mite, maple bladder gall mite, alder erineum mite, redberry mite, cotton blister mite, blueberry bud mite, pink tea rust mite, ribbed tea mite, grey citrus mite, sweet potato rust mite, horse chestnut rust mite, citrus rust mite, apple rust mite, grape rust mite, pear rust mite, flat needle sheath pine mite, wild rose bud and fruit mite, dryberry mite, mango rust mite, azalea rust mite, plum rust mite, peach silver mite, apple rust mite, tomato russet mite, pink citrus rust mite, cereal rust mite, rice rust mite and combinations thereof.

14. The method of claim 12, wherein the Diabrotica spp. is selected from the group consisting of Banded cucumber beetle (Diabrotica balteata), Northern corn rootworm (Diabrotica barberi), Southern corn rootworm (Diabrotica undecimpunctata howardi), Western cucumber beetle (Diabrotica undecimpunctata tenella), Western spotted cucumber beetle (Diabrotica undecimpunctata undecimpunctata), Western corn rootworm (Diabrotica virgifera virgifera), Mexican corn rootworm (Diabrotica virgifera zeae) and combinations thereof.

15. The method of claim 8, wherein the plant disease is caused by a fungus.

16. The method of claim 8, wherein the plant disease is a leaf blotch disease or a leaf wilt disease.

17. The method of claim 16, wherein the plant disease is caused by Venturia sp. or Mycosphaerella sp.

18. The method of claim 8, wherein the plant disease is a mildew or a rust disease.

19. The method of claim 18, wherein the mildew is powdery mildew or downy mildew.

20. The method of claim 19, wherein the powdery mildew is caused by a pathogen selected from the group consisting of Blumeria sp., Podosphaera sp., Sphaerotheca sp., and Uncinula sp.

21. The method of claim 20, wherein the pathogen is Podosphaera xanthii.

22. The method of claim 18 wherein the rust disease is selected from the group consisting of wheat leaf rust leaf rust caused by Puccinia triticina, leaf rust of barley caused by Puccinia hordei, leaf rust of rye caused by Puccinia recondita, brown leaf rust, crown rust, and stem rust.

23. A method of treating a plant to control a mildew, leaf blotch disease or a leaf wilt disease, wherein the method comprises applying a composition comprising Streptomyces microflavus strain NRRL B-50550 or a phytophagous-miticidal and/or fungcidal mutant strain derived therefrom, to the plant, to a part of the plant and/or to a locus of the plant.

24. The method of claim 23, wherein the composition is a fermentation product of the strain.

25. The method of claim 23, wherein the method comprises applying the composition to foliar plant parts.

26. The method of claim 23, wherein the leaf blotch disease or leaf wilt disease is caused by Venturia sp. or Mycosphaerella sp.

27. The method of claim 23, wherein the mildew is caused by a pathogen selected from the group consisting of Blumeria sp., Podosphaera sp., Sphaerotheca sp., and Uncinula sp.

28. The method of claim 27, wherein the pathogen is Podosphaera xanthii.

29. A method of producing a fermentation broth of a gougerotin-producing Streptomyces strain, wherein the fermentation broth contains at least about 2 g/L gougerotin, the method comprising:
a) screening a collection of Streptomyces strains to identify at least one gougerotin-producing Streptomyces strain;
b) generating a plurality of mutant strains from the at least one gougerotin-producing Streptomyces strain;
c) screening the plurality of mutant strains to identify at least one mutant strain that produces a fermentation broth containing at least about 2 g/L gougerotin; and
d) cultivating the at least one mutant strain in a culture medium containing a digestible carbon source and a digestible nitrogen source under aerobic conditions.

30. The method of claim 29, wherein the at least one gougerotin-producing Streptomyces strain comprises Streptomyces microflavus NRRL B-50550 and/or Streptomyces microflavus strain No. 091013-02.

31. A method of producing a fermentation broth of a gougerotin producing Streptomyces strain, wherein the fermentation broth contains at least about 2 g/L gougerotin, the method comprising:
a) generating a plurality of mutant strains of Streptomyces microflavus NRRL B-50550 and/or Streptomyces microflavus strain No. 091013-02;
b) screening the plurality of mutant strains to identify at least one mutant strain that produces a fermentation broth containing at least about 2 g/L gougerotin; and
c) cultivating the at least one mutant strain in a culture medium containing a digestible carbon source and a digestible nitrogen source under aerobic conditions.

32. The method of claim 29, wherein the at least one mutant strain undergoes one or more additional cycles of mutagenesis and after each cycle of mutagenesis only mutant strains producing a fermentation broth containing at least about 2 g/L gougerotin continue to a subsequent cycle of mutagenesis.

33. The method of claim 29, wherein the mutant strains are generated by genome shuffling.

34. The method of claim 29, wherein the mutant strains are not generated by chemical mutagenesis with N-methyl-N′-nitro-N-nitrosoguanidine (NTG).

35. The method of claim 29, wherein the culture medium comprises glycine, L-glutamic acid, L-glutamine, L-aspartic acid, L-serine, or a mixture thereof.

36. The method of claim 29, wherein the fermentation broth contains at least about 3 g/L, at least about 4 g/L, at least about 5 g/L, at least about 6 g/L, at least about 7 g/L or at least about 8 g/L gougerotin.

37. A fermentation broth made by the method of claim 29.
